(19) United States
(12) Patent Application Publication
(10) Pub. No.: US 2024/0138743 A1
(43) Pub. Date: May 2, 2024

(54) PREMATURE VENTRICULAR CONTRACTION (PVC) DETECTION USING AN ARTIFICIAL INTELLIGENCE MODEL

(71) Applicant: Medtronic, Inc., Minneapolis, MN (US)

(72) Inventors: Gautham Rajagopal, Minneapolis, MN (US); Shantanu Sarkar, Roseville, MN (US)

(21) Appl. No.: 18/050,814

(22) Filed: Oct. 28, 2022

(51) Int. Cl. A61B 5/349; A61B 5/29; A61B 5/352; G16H 50/20

(52) U.S. Cl. CPC A61B 5/349; A61B 5/29; A61B 5/352; G16H 50/20

(57) ABSTRACT

An example medical system includes a plurality of electrodes configured to sense a cardiac electrogram of a patient; and processing circuitry configured to: perform feature extraction of the sensed cardiac electrogram to extract a plurality of features from the sensed cardiac electrogram; convert one or more of the plurality of extracted features to a respective feature image; apply a machine learning model, trained using cardiac electrogram data for a plurality of patients, to an image of the sensed cardiac electrogram and at least the respective feature image to determine whether one or more particular heartbeats in the sensed cardiac electrogram indicate a premature ventricular contraction (PVC) beat; and in response to the determination that the one or more particular heartbeats indicates a PVC beat, output a classification that the one or more particular heartbeats is a PVC beat.

### FIELD

The disclosure relates generally to medical device systems and, more particularly, medical device systems configured to detect premature ventricular contractions (PVCs).

### BACKGROUND

Medical devices may be used to monitor physiological signals of a patient. For example, some medical devices are configured to sense cardiac electrogram (EGM) signals indicative of the electrical activity of the heart via electrodes. Some medical devices may be configured to deliver a therapy in conjunction with or separate from the monitoring of physiological signals.

PVCs are premature heartbeats originating from the ventricles of the heart. PVCs are premature because they occur before the regular heartbeat originating from the sinoatrial node. During a PVC event, the ventricles electrically discharge and contract prematurely before the normal electrical discharge arrives from the sinoatrial node. PVCs may occur in healthy individuals. PVCs may be caused by caffeine, smoking, alcohol consumption, stress, exhaustion, pharmacological toxicity, electrolyte imbalance, lack of oxygen, and heart attack as examples. Common symptoms associated with PVCs include palpitations, dizziness, fatigue, dyspnea, chest pain, and lightheadedness. PVCs are normally considered benign, but may potentially cause cardiomyopathy, ventricular arrythmias, and heart failure.

Management strategies for PVC induced cardiomyopathy include medical therapy and catheter ablation, with an increasing role for catheter ablation in view of the potential for permanent suppression of PVCs. Ablation to suppress PVCs may lead to improvement of left ventricular systolic dysfunction (LVSD) and normalization of left ventricular ejection fraction (LVEF). PVC burden, i.e., a quantification of the amount of PVCs over a period of time, can be an independent predictor of PVC induced cardiomyopathy. Presently, 24-hour Holter monitoring is the most commonly used method to determine PVC burden.

### SUMMARY

In general, this disclosure is directed to techniques for detecting PVCs using a cardiac electrogram (EGM) sensed by a medical device to, for example, facilitate a determination of PVC burden. More particularly, the disclosure is directed to techniques for evaluating a cardiac EGM to determine whether a particular heartbeat is a PVC. Processing circuitry may determine that a particular heartbeat is a PVC based on extracting particular features from the cardiac EGM, convert the extracted features to a respective feature image, and applying an image of the cardiac EGM and the one or more feature images to an artificial intelligence model, such as a machine learning model or other suitable model. In some examples, processing circuitry may weight and/or normalize a cardiac EGM signal image and feature images and apply the weighted and/or normalized cardiac EGM signal image and feature images to a machine learning model to determine whether a particular heartbeat is a PVC.

For some medical devices, e.g., those utilizing external, subcutaneous, or other extra-vascular electrodes, the location and orientation of the electrodes used to sense the cardiac EGM relative to the heart and other tissue may vary between patients, and within a given patient over time. The criteria used by the processing circuitry to determine whether a particular heartbeat is a PVC according to the techniques of this disclosure may improve sensitivity and/or specificity of PVC detection. In some examples, improving the sensitivity and/or specificity of PVC detection may facilitate more accurate determinations of PVC burden, cardiac wellness, and risk of sudden cardiac death, and may lead to clinical interventions to suppress PVCs such as medications and PVC ablations.

Unlike conventional PVC detection systems, the techniques and systems of this disclosure may use a machine learning model to more accurately determine whether one or more particular heartbeats in the sensed cardiac electrogram indicate a premature ventricular contraction (PVC) beat. In some examples, the machine learning model is trained with a set of training instances, where one or more of the training instances comprise data that indicate relationships between various EGM features (including features for particular heartbeats) and classifications whether a PVC occurred or not for such features. Because the machine learning model is trained with potentially thousands or millions of training instances, the machine learning model may reduce the amount of classification error in classifying one or more heartbeats as a PVC when compared to conventional PVC detection systems. Additionally, the techniques and systems of this disclosure may be implemented in an implantable medical device (IMD) that can continuously and/or periodically sense EGM without human intervention while subcutaneously implanted in a patient over months or years and perform millions of operations per second on patient EGM data to identify PVCs with a machine learning model. Using techniques of this disclosure in an IMD may be advantageous when a physician cannot be continuously present with the patient over weeks or months to evaluate EGM and/or where performing millions of operations on weeks or months of EGM data could not practically be performed in the mind of a physician with techniques of this disclosure that use a machine learning model.

Reducing classification errors for PVCs with a machine learning model implementing techniques of this disclosure may provide one or more technical and clinical advantages. In some examples, a medical system converting extracted feature(s) of the EGM data to respective feature image(s) and applying a trained machine learning model to an image of the sensed EGM and the converted feature image(s) may help the determination that a particular heartbeat indicates a PVC beat to have higher specificity and sensitivity. For example, when a system or computing device determining that a particular heartbeat indicates a PVC beat has higher specificity and sensitivity, a number of false positives may be reduced. For instance, using a machine learning model as described in this disclosure may require less power because reducing the number of false positives may require fewer communications of PVCs from the IMD to other computing devices and improve the longevity of the IMD when implanted subcutaneously in the patient for use over multiple years. In some examples, using a machine learning model as described in this disclosure that may result in higher specificity and sensitivity of determining whether a particular heartbeat indicates a PVC beat. This higher specificity and sensitivity may increase reliability of another device, user, and/or clinician on the accuracy of determining whether a particular heartbeat indicates a PVC beat. In some examples, this improved reliability on the accuracy of determining whether a particular heartbeat indicates a PVC may result in improved usefulness of the system or computing device as a clinician, user, or other computing device may not use and/or rely upon determinations that are not at or above a specificity and sensitivity threshold. Systems and techniques of this disclosure using a machine learning model may also more flexibly classify or predict PVCs from particular portions of an EGM by eliminating the need to configure explicit rule sets in an IMD that may otherwise be too expansive in size to practically implement and process for each new portion of an EGM sensed for a patient. Furthermore, reducing the number of PVC false positives when using techniques of this disclosure may decrease the clinical burden on physicians and other caregivers to review and identify true positive PVCs. With more accurate PVC classifications provided by the machine learning model used with techniques of this disclosure, physicians and caregivers may also provide better-tailored care, therapies, and interventions for the patient experiencing PVC.

In one example, a medical system comprises a plurality of electrodes configured to sense a cardiac electrogram of a patient; and processing circuitry configured to: perform feature extraction of the sensed cardiac electrogram to extract a plurality of features from the sensed cardiac electrogram; convert one or more of the plurality of extracted features to a respective feature image; apply a machine learning model, trained using cardiac electrogram data for a plurality of patients, to an image of the sensed cardiac electrogram and at least the respective feature image to determine whether one or more particular heartbeats in the sensed cardiac electrogram indicate a premature ventricular contraction (PVC) beat; and in response to the determination that the one or more particular heartbeats indicates a PVC beat, output a classification that the one or more particular heartbeats is a PVC beat.

In another example, a computer device comprises a memory; and processing circuitry coupled to the memory, the processing circuitry being configured to: receive a sensed cardiac electrogram of a patient; perform feature extraction of the sensed cardiac electrogram to extract a plurality of features from the sensed cardiac electrogram; convert one or more of the plurality of extracted features to a respective feature image; apply a machine learning model, trained using cardiac electrogram data for a plurality of patients, to an image of the sensed cardiac electrogram and at least the respective feature image to determine whether one or more particular heartbeats in the sensed cardiac electrogram indicate a premature ventricular contraction (PVC) beat; and in response to the determination that the one or more particular heartbeats indicates a PVC beat, output a classification that the one or more particular heartbeats is a PVC beat.

In another example, a method comprises receiving a sensed cardiac electrogram of a patient; performing feature extraction of the sensed cardiac electrogram to extract a plurality of features from the sensed cardiac electrogram; converting one or more of the plurality of extracted features to a respective feature image; applying a machine learning model, trained using cardiac electrogram data for a plurality of patients, to an image of the sensed cardiac electrogram and at least the respective feature image to determine whether one or more particular heartbeats in the sensed cardiac electrogram indicate a premature ventricular contraction (PVC) beat; and in response to the determination that the one or more particular heartbeats indicates a PVC beat, outputting a classification that the one or more particular heartbeats is a PVC beat.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates the environment of an example medical system in conjunction with a patient.

FIG. 2 is a functional block diagram illustrating an example configuration of the implantable medical device (IMD) of the medical system of FIG. 1.

FIG. 3 is a conceptual side-view diagram illustrating an example configuration of the IMD of FIGS. 1 and 2.

FIG. 4A is a conceptual side-view diagram illustrating an example configuration of the IMD of FIGS. 1-3.

FIG. 4B is a schematic diagram illustrating an example configuration of the IMD of FIG. 4A.

FIG. 4C is a conceptual side-view diagram illustrating an example configuration of the IMD of FIGS. 1-3.

FIG. 5 is a functional block diagram illustrating an example configuration of the external device of FIGS. 1-4.

FIG. 6 is a block diagram illustrating an example system that includes an access point, a network, external computing devices, such as a server, and one or more other computing devices, which may be coupled to the IMD and external device of FIGS. 1-5.

FIG. 7A is a graph illustrating a cardiac EGM, in accordance with some examples of the current disclosure.

FIG. 7B is a graph illustrating a plurality of correlation coefficients being determined from a cardiac EGM, in accordance with some examples of the current disclosure.

FIG. 7C is a graph illustrating a feature image of a plurality of correlation coefficients, in accordance with some examples of the current disclosure.

FIG. 8A is a graph illustrating a plurality of RR intervals being determined from a cardiac EGM, in accordance with some examples of the current disclosure.

FIG. 8B is a graph illustrating a feature image of a plurality of RR intervals, in accordance with some examples of the current disclosure.

FIG. 9 is a graph illustrating weighting of images based on the cardiac EGM, in accordance with some examples of the current disclosure.

FIG. 10 is a graph illustrating weighted images of extracted features from a cardiac EGM of a normal heartbeat, in accordance with some examples of the current disclosure.

FIGS. 11A and 11B are graphs illustrating weighted images of extracted features from a cardiac EGM that did not have the axes normalized, in accordance with some examples of the current disclosure.

FIGS. 12A and 12B are graphs illustrating normalized and weighted images of extracted features from a cardiac EGM, in accordance with some examples of the current disclosure.

FIG. 13 is a chart illustrating the results of normalized and weighted images of extracted features from a cardiac EGM being applied to a machine learning model, in accordance with some examples of the current disclosure.

FIG. 14 is a chart illustrating the confusion matrix on the validation dataset of FIG. 13, in accordance with some examples of the current disclosure.

FIG. 15 is a conceptual diagram illustrating an example machine learning model configured to determine whether a particular heartbeat in the sensed cardiac electrogram is a PVC beat and/or a score indicative of whether a particular heartbeat may be a PVC beat.

FIG. 16 is a conceptual diagram illustrating an example training process for an artificial intelligence model, in accordance with examples of the current disclosure.

FIG. 17 is a flow diagram illustrating an example technique for operating a system to determine whether a particular heartbeat in the sensed cardiac electrogram is a PVC beat.

Like reference characters denote like elements throughout the description and figures.

### DETAILED DESCRIPTION

A variety of types of medical devices sense cardiac EGMs. In some examples, EGMs may also include electrocardiogram (ECGs or EKGs). Some medical devices that sense cardiac EGMs are non-invasive, e.g., using a plurality of electrodes placed in contact with external portions of the patient, such as at various locations on the skin of the patient. The electrodes used to monitor the cardiac EGM in these non-invasive processes may be attached to the patient using an adhesive, strap, belt, or vest, as examples, and electrically coupled to a monitoring device, such as an electrocardiograph, Holter monitor, or other electronic device. The electrodes are configured to sense electrical signals associated with the electrical activity of the heart or other cardiac tissue of the patient, and to provide these sensed electrical signals to the electronic device for further processing and/or display of the electrical signals. The non-invasive devices and methods may be utilized on a temporary basis, for example to monitor a patient during a clinical visit, such as during a doctor's appointment, or for example for a predetermined period of time, for example for one day (twenty-four hours), or for a period of several days.

External devices that may be used to non-invasively sense and monitor cardiac EGMs include wearable devices with electrodes configured to contact the skin of the patient, such as patches, watches, or necklaces. One example of a wearable physiological monitor configured to sense a cardiac EGM is the SEEQ™ Mobile Cardiac Telemetry System, available from Medtronic plc, of Dublin, Ireland. Such external devices may facilitate relatively longer-term monitoring of patients during normal daily activities, and may periodically transmit collected data to a network service, such as the Medtronic Carelink™ Network.

Some implantable medical devices (IMDs) also sense and monitor cardiac EGMs. The electrodes used by IMDs to sense cardiac EGMs are typically integrated with a housing of the IMD and/or coupled to the IMD via one or more elongated leads. Example IMDs that monitor cardiac EGMs include pacemakers and implantable cardioverter-defibrillators, which may be coupled to intravascular or extravascular leads, as well as pacemakers with housings configured for implantation within the heart, which may be leadless. An example of pacemaker configured for intracardiac implantation is the Micra™ Transcatheter Pacing System, available from Medtronic plc. Some IMDs that do not provide therapy, e.g., implantable patient monitors, sense cardiac EGMs. One example of such an IMD is the Reveal LINQ™ and LINO II™ Insertable Cardiac Monitors (ICMs), available from Medtronic plc, which may be inserted subcutaneously. Such IMDs may facilitate relatively longer-term monitoring of patients during normal daily activities, and may periodically transmit collected data to a network service, such as the Medtronic Carelink™ Network.

Any medical device configured to sense a cardiac EGM via implanted or external electrodes, including the examples identified herein, may implement the techniques of this disclosure for evaluating a cardiac EGM to determine whether the particular heartbeat in the cardiac EGM is a PVC. For example, features may be extracted from the cardiac EGM, the extracted features may be modified to a respective feature image, and an image of the EGM and the one or more feature images may be applied to a machine learning model to determine whether a particular heartbeat is a PVC which leads to accurate determinations of PVC with high specificity and sensitivity. The techniques of this disclosure for determining whether a particular heartbeat is a PVC may facilitate determinations of PVC burden, cardiac wellness, and risk of sudden cardiac death, and may lead to clinical interventions to suppress PVCs such as medications and PVC ablations.

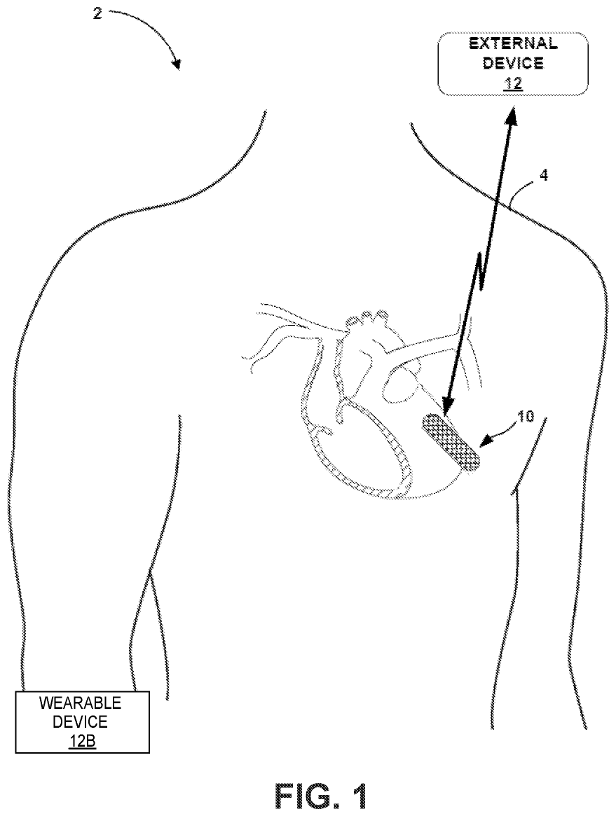

FIG. 1 illustrates the environment of an example medical system 2 in conjunction with a patient 4, in accordance with one or more techniques of this disclosure. The example techniques may be used with an IMD 10, which may be in wireless communication with at least one of external device 12 and other devices not pictured in FIG. 1. In some examples, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. IMD 10 includes a plurality of electrodes (not shown in FIG. 1), and is configured to sense a cardiac EGM via the plurality of electrodes. In some examples, IMD 10 takes the form of the Reveal LINQ™ or LINQ II ICM™, or another ICM similar to, e.g., a version or modification of, the LINQ™ ICMs.

External device 12 may be a computing device with a display viewable by the user and an interface for providing input to external device 12 (i.e., a user input mechanism). In some examples, external device 12 may be a notebook computer, tablet computer, workstation, one or more servers, cellular phone, smartphone, personal digital assistant, or another computing device that may run an application that enables the computing device to interact with IMD 10. External device 12 is configured to communicate with IMD 10 and, optionally, another computing device (not illustrated in FIG. 1), via wireless communication. External device 12, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth® specification sets, or other communication technologies operable at ranges greater than near-field communication technologies).

In some examples, external device 12 may be or additionally include wearable computing device 12B. Wearable computing device 12B may include electrodes and other sensors to sense physiological signals of patient 4, and may collect and store physiological data and detect episodes based on such signals. Wearable computing device 12B may be incorporated into the apparel of patient 4, such as within clothing, shoes, eyeglasses, a watch or wristband, a hat, etc. In some examples, computing device 12B is a smartwatch or other accessory or peripheral for external device 12, for example when external device 12 is a smartphone or tablet.

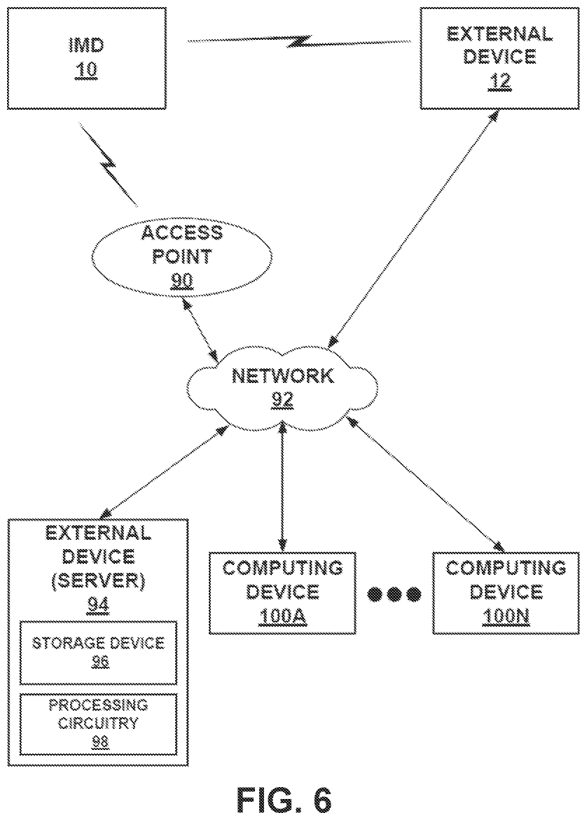

External device 12 may be used to configure operational parameters for IMD 10. External device 12 may be used to retrieve data from IMD 10. The retrieved data may include values of physiological parameters measured by IMD 10, indications of episodes of arrhythmia or other maladies detected by IMD 10, and physiological signals recorded by IMD 10. For example, external device 12 may retrieve information related to detection of PVCs by IMD 10, such as a count or other quantification of PVCs, e.g., over a time period since the last retrieval of information by external device. External device 12 may also retrieve cardiac EGM segments recorded by IMD 10, e.g., due to IMD 10 determining that an episode of arrhythmia or another malady occurred during the segment, or in response to a request to record the segment from patient 4 or another user. As discussed in greater detail below with respect to FIG. 6, one or more remote computing devices may interact with IMD 10 in a manner similar to external device 12, e.g., to program IMD 10 and/or retrieve data from IMD 10, via a network.

Processing circuitry of medical system 2, e.g., of IMD 10, external device 12, and/or of one or more other computing devices, may be configured to perform the example techniques of this disclosure for determining whether a particular heartbeat is a PVC. In some examples, processing circuitry of medical system 2 may be configured to perform the example techniques of this disclosure to determine whether a cardiac EGM includes PVC couplets, triplets, and/or non-sustained ventricular tachycardia (VT). In some examples, the processing circuitry of medical system 2 analyzes a cardiac EGM sensed by IMD 10 to determine whether a particular heartbeat is a PVC based on whether the particular heartbeat and adjacent heartbeats in the cardiac EGM, e.g., comparisons between these heartbeats, satisfy a plurality of criteria. In some examples, the processing circuitry of medical system 2 analyzes a cardiac EGM sensed by IMD 10 to determine whether a particular pair or triplet of heartbeats are a respective PVC couplet or triplet based on whether the particular pair of heartbeats or triplet of heartbeats and adjacent heartbeats to the pair or triplet of heartbeats in in the cardiac EGM, e.g., comparisons between these heartbeats, satisfy a plurality of criteria. The criteria may include noise criteria, inter-depolarization interval (e.g., R-R interval) criteria, and/or morphological criteria, as described in greater detail below. Although described in the context of examples in which IMD 10 that senses the cardiac EGM comprises an insertable cardiac monitor, example systems including one or more implantable or external devices of any type configured to sense a cardiac EGM may be configured to implement the techniques of this disclosure.

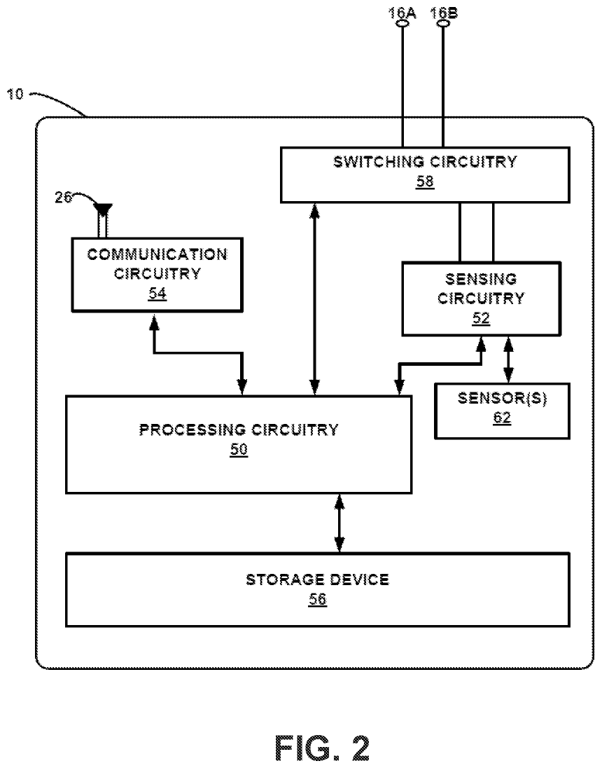

FIG. 2 is a functional block diagram illustrating an example configuration of IMD 10 of FIG. 1 in accordance with one or more techniques described herein. In the illustrated example, IMD 10 includes electrodes 16A and 16B (collectively “electrodes 16”), antenna 26, processing circuitry 50, sensing circuitry 52, communication circuitry 54, storage device 56, switching circuitry 58, and sensors 62. Although the illustrated example includes two electrodes 16, IMDs including or coupled to more than two electrodes 16 may implement the techniques of this disclosure in some examples.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16 via switching circuitry 58, e.g., to select the electrodes 16 and polarity, referred to as the sensing vector, used to sense a cardiac EGM, as controlled by processing circuitry 50. Sensing circuitry 52 may sense signals from electrodes 16, e.g., to produce a cardiac EGM, in order to facilitate monitoring the electrical activity of the heart. Sensing circuitry 52 also may monitor signals from sensors 62, which may include one or more accelerometers, pressure sensors, and/or optical sensors, as examples. In some examples, signals received from sensors 62 may be used in addition to a sensed cardiac EGM to detect whether a particular heartbeat is a PVC. In some examples, sensing circuitry 52 may include one or more filters and amplifiers for filtering and amplifying signals received from electrodes 16 and/or sensors 62.

Sensing circuitry 52 and/or processing circuitry 50 may be configured to detect cardiac depolarizations (e.g., P-waves of atrial depolarizations or R-waves of ventricular depolarizations) when the cardiac EGM amplitude crosses a sensing threshold. For cardiac depolarization detection, sensing circuitry 52 may include a rectifier, filter, amplifier, comparator, and/or analog-to-digital converter, in some examples. In some examples, sensing circuitry 52 may output an indication to processing circuitry 50 in response to sensing of a cardiac depolarization. In this manner, processing circuitry 50 may receive detected cardiac depolarization indicators corresponding to the occurrence of detected R-waves and P-waves in the respective chambers of heart. Processing circuitry 50 may use the indications of detected R-waves and P-waves for determining inter-depolarization intervals, heart rate, and detecting arrhythmias, such as tachyarrhythmias and asystole.

Sensing circuitry 52 may also provide one or more digitized cardiac EGM signals to processing circuitry 50 for analysis, e.g., for use in cardiac rhythm discrimination, and/or for analysis to determine whether one or more PVC detection criteria are satisfied according to the techniques of this disclosure. In some examples, processing circuitry 50 may store the digitized cardiac EGM in storage device 56. Processing circuitry 50 of IMD 10, and/or processing circuitry of another device that retrieves data from IMD 10, may analyze the cardiac EGM to determine whether one or more PVC detection criteria are satisfied according to the techniques of this disclosure.

Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network. Antenna 26 and communication circuitry 54 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Storage device 56 may store, as examples, programmed values for one or more operational parameters of IMD 10 and/or data collected by IMD 10 for transmission to another device using communication circuitry 54. Data stored by storage device 56 and transmitted by communication circuitry 54 to one or more other devices may include PVC detection quantifications and/or digitized cardiac EGMs, as examples.

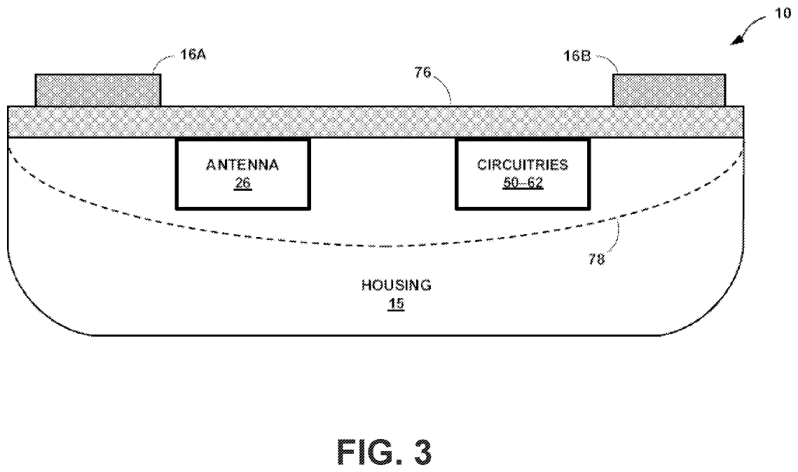

FIG. 3 is a conceptual side-view diagram illustrating an example configuration of IMD 10 of FIGS. 1 and 2. In the example shown in FIG. 3, IMD 10 may include a leadless, subcutaneously-implantable monitoring device having a housing 15 and an insulative cover 76. Electrode 16A and electrode 16B may be formed or placed on an outer surface of cover 76. Circuitries 50-62, described above with respect to FIG. 2, may be formed or placed on an inner surface of cover 76, or within housing 15. In the illustrated example, antenna 26 is formed or placed on the inner surface of cover 76, but may be formed or placed on the outer surface in some examples. In some examples, one or more of sensors 62 may be formed or placed on the outer surface of cover 76. In some examples, insulative cover 76 may be positioned over an open housing 15 such that housing 15 and cover 76 enclose antenna 26 and circuitries 50-62, and protect the antenna and circuitries from fluids such as body fluids.

One or more of antenna 26 or circuitries 50-62 may be formed on the inner side of insulative cover 76, such as by using flip-chip technology. Insulative cover 76 may be flipped onto a housing 15. When flipped and placed onto housing 15, the components of IMD 10 formed on the inner side of insulative cover 76 may be positioned in a gap 78 defined by housing 15. Electrodes 16 may be electrically connected to switching circuitry 58 through one or more vias (not shown) formed through insulative cover 76. Insulative cover 76 may be formed of sapphire (i.e., corundum), glass, parylene, and/or any other suitable insulating material. Housing 15 may be formed from titanium or any other suitable material (e.g., a biocompatible material). Electrodes 16 may be formed from any of stainless steel, titanium, platinum, iridium, or alloys thereof. In addition, electrodes 16 may be coated with a material such as titanium nitride or fractal titanium nitride, although other suitable materials and coatings for such electrodes may be used.

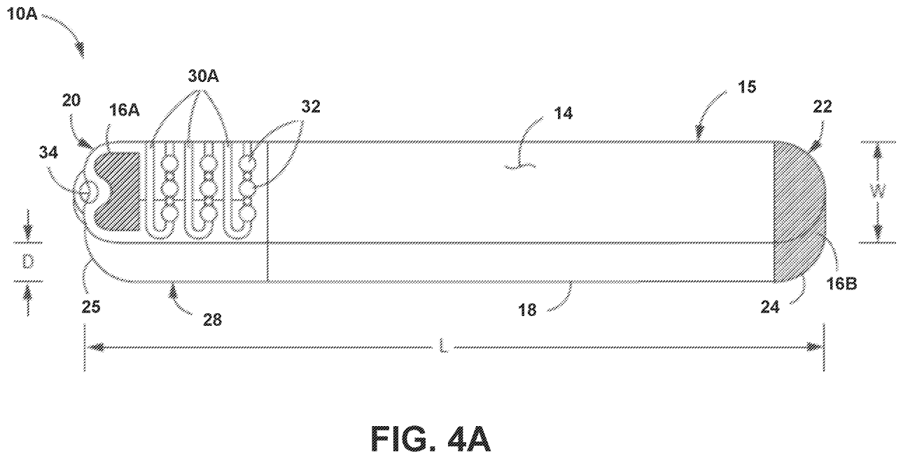

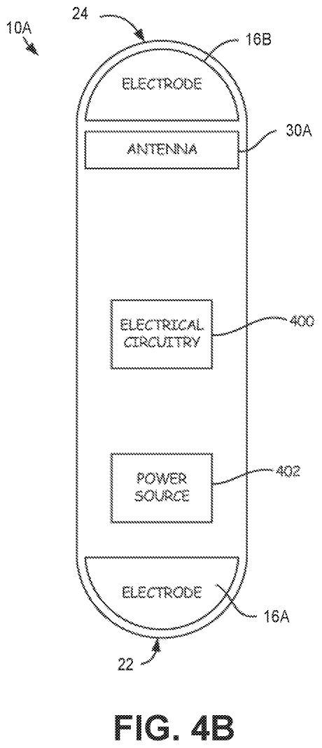

FIG. 4A is a conceptual drawing illustrating an IMD 10A, which may be an example configuration of IMD 10 of FIGS. 1-3 as an ICM. In the example shown in FIG. 4A, IMD 10A may be embodied as a monitoring device having housing 15, proximal electrode 16A and distal electrode 16B. Housing 15 may further comprise first major surface 14, second major surface 18, proximal end 20, and distal end 22. Housing 15 encloses electronic circuitry located inside the IMD 10A and protects the circuitry contained therein from body fluids. Electrical feedthroughs provide electrical connection of electrodes 16A and 16B.

In the example shown in FIG. 4A, IMD 10A is defined by a length L, a width W and thickness or depth D and is in the form of an elongated rectangular prism wherein the length L is much larger than the width W, which in turn is larger than the depth D. In one example, the geometry of the IMD 10A—in particular a width W greater than the depth D—is selected to allow IMD 10A to be inserted under the skin of the patient using a minimally invasive procedure and to remain in the desired orientation during insertion. For example, the device shown in FIG. 4A includes radial asymmetries (notably, the rectangular shape) along the longitudinal axis that maintains the device in the proper orientation following insertion. For example, the spacing between proximal electrode 64 and distal electrode 66 may range from 30 millimeters (mm) to 55 mm, 35 mm to 55 mm, and from 40 mm to 55 mm and may be any range or individual spacing from 25 mm to 60 mm. In addition, IMD 10A may have a length L that ranges from 30 mm to about 70 mm. In other examples, the length L may range from 5 mm to 60 mm, 15 mm to 50 mm, 40 mm to 60 mm, 45 mm to 60 mm and may be any length or range of lengths between about 5 mm and about 80 mm. In addition, the width W of major surface 14 may range from 5 mm to 15 mm, 3 mm to 10 mm, and may be any single or range of widths between 3 mm and 15 mm. The thickness of depth D of IMD 10A may range from 2 mm to 9 mm. In other examples, the depth D of IMD 10A may range from 2 mm to 5 mm, may range from 5 mm to 15 mm, and may be any single or range of depths from 2 mm to 15 mm. In addition, IMD 10A according to an example of the present disclosure is has a geometry and size designed for ease of implant and patient comfort. Examples of IMD 10A described in this disclosure may have a volume of three cubic centimeters (cm) or less, 1.5 cubic cm or less or any volume between three and 1.5 cubic centimeters.

In the example shown in FIG. 4A, once inserted within the patient, the first major surface 14 faces outward, toward the skin of the patient while the second major surface 18 is located opposite the first major surface 14. In addition, in the example shown in FIG. 4A, proximal end 20 and distal end 22 are rounded to reduce discomfort and irritation to surrounding tissue once inserted under the skin of the patient. IMD 10A, including instrument and method for inserting IMD 10 is described, for example, in U.S. Patent Publication No. 2014/0276928, incorporated herein by reference in its entirety.

Proximal electrode 16A and distal electrode 16B are used to sense cardiac signals, e.g. EGM signals, intra-thoracically or extra-thoracically, which may be sub-muscularly or subcutaneously. EGM signals may be stored in a memory of IMD 10A, and data may be transmitted via integrated antenna 30A to another medical device, which may be another implantable device or an external device, such as external device 12. In some example, electrodes 16A and 16B may additionally or alternatively be used for sensing any bio-potential signal of interest, which may be, for example, an EGM, EEG, EMG, or a nerve signal, from any implanted location.

In the example shown in FIG. 4A, proximal electrode 16A is in close proximity to the proximal end 20 and distal electrode 16B is in close proximity to distal end 22. In this example, distal electrode 16B is not limited to a flattened, outward facing surface, but may extend from first major surface 14 around rounded edges 24 and/or end surface 25 and onto the second major surface 18 so that the electrode 16B has a three-dimensional curved configuration. In some examples, electrode 16B is an uninsulated portion of a metallic, e.g., titanium, part of housing 15.

In the example shown in FIG. 4A, proximal electrode 16A is located on first major surface 14 and is substantially flat, and outward facing. However, in other examples proximal electrode 16A may utilize the three dimensional curved configuration of distal electrode 16B, providing a three dimensional proximal electrode (not shown in this example). Similarly, in other examples distal electrode 16B may utilize a substantially flat, outward facing electrode located on first major surface 14 similar to that shown with respect to proximal electrode 16A.

The various electrode configurations allow for configurations in which proximal electrode 16A and distal electrode 16B are located on both first major surface 14 and second major surface 18. In other configurations, such as that shown in FIG. 4A, only one of proximal electrode 16A and distal electrode 16B is located on both major surfaces 14 and 18, and in still other configurations both proximal electrode 16A and distal electrode 16B are located on one of the first major surface 14 or the second major surface 18 (e.g., proximal electrode 16A located on first major surface 14 while distal electrode 16B is located on second major surface 18). In another example, IMD 10A may include electrodes on both major surface 14 and 18 at or near the proximal and distal ends of the device, such that a total of four electrodes are included on IMD 10A. Electrodes 16A and 16B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 4A, proximal end 20 includes a header assembly 28 that includes one or more of proximal electrode 16A, integrated antenna 30A, anti-migration projections 32, and/or suture hole 34. Integrated antenna 30A is located on the same major surface (i.e., first major surface 14) as proximal electrode 16A and is also included as part of header assembly 28. Integrated antenna 30A allows IMD 10A to transmit and/or receive data. In other examples, integrated antenna 30A may be formed on the opposite major surface as proximal electrode 16A, or may be incorporated within the housing 15 of IMD 10A. In the example shown in FIG. 4A, anti-migration projections 32 are located adjacent to integrated antenna 30A and protrude away from first major surface 14 to prevent longitudinal movement of the device. In the example shown in FIG. 4A, anti-migration projections 32 include a plurality (e.g., nine) small bumps or protrusions extending away from first major surface 14. As discussed above, in other examples anti-migration projections 32 may be located on the opposite major surface as proximal electrode 16A and/or integrated antenna 30A. In addition, in the example shown in FIG. 4A, header assembly 28 includes suture hole 34, which provides another means of securing IMD 10A to the patient to prevent movement following insertion. In the example shown, suture hole 34 is located adjacent to proximal electrode 16A. In one example, header assembly 28 is a molded header assembly made from a polymeric or plastic material, which may be integrated or separable from the main portion of IMD 10A.

FIG. 4B is a functional schematic diagram of IMD 10A as shown in FIG. 4A according to an embodiment of the present disclosure. IMD 10A may include housing 15, proximal electrode 16B located at proximal end 24, distal electrode 16A located at distal end 22, integrated antenna 30A, electrical circuitry 400 and power source 402. In particular, electrical circuitry 400 is coupled to proximal electrode 16B and distal electrode 16A to sense cardiac signals and monitor events. Electrical circuitry 400 may also connected to transmit and receive communications via integrated antenna 30A. Power source 402 provides power to electrical circuitry 400, as well as to any other components that require power. Power source 402 may include one or more energy storage devices, such as one or more rechargeable or non-rechargeable batteries. In some examples, electrical circuitry 400 includes processing circuitry 50 and storage device 56, such as a memory, as shown in FIG. 2, the memory being operatively coupled to the processing circuitry 56 and configured to store a machine learning model.

In the example shown in FIG. 4B, electrical circuitry 400 may receive raw EGM signals monitored by proximal electrode 16B and distal electrode 16A. Electrical circuitry 400 may include components/modules for converting the raw EGM signal to a processed EGM signal that can be analyzed to detect sense events. Although not shown, electrical circuitry 400 may include any discrete and/or integrated electronic circuit components that implement analog and/or digital circuits capable of producing the functions described for analyzing EGM signals to detect/verify PVC. For example, the electrical circuitry 400 may include analog circuits, e.g., pre-amplification circuits, filtering circuits, and/or other analog signal conditioning circuits. The modules may also include digital circuits, e.g., digital filters, combinational or sequential logic circuits, state machines, integrated circuits, a processor (shared, dedicated, or group) that executes one or more software or firmware programs, memory devices, or any other suitable components or combination thereof that provide the described functionality.

In one example, electrical circuitry 400 includes a sensing unit for monitoring the EGM signal detected by the respective proximal and distal electrodes 16A and 16B, respectively. In one example, electrical circuitry 400 includes processing circuitry 50 that is utilized to receive information regarding sensed events and implements one or more algorithms for determining whether a PVC beat has occurred. In addition, the analog voltage signals received from electrodes 16A and 16B may be passed to analog-to-digital (A/D) converters included in the electrical circuitry 400, and stored in a memory unit (not shown) included as part of electrical circuitry 400 for subsequent analysis with firmware executed by the processor included as part of electrical circuitry 400.

In some examples, housing 15 may be a hermetically-sealed housing configured for subcutaneous implantation within the patient, wherein at least the power source 402, memory, and processing circuitry 50 are within the hermetically-sealed case.

FIG. 4C is a perspective drawing illustrating another IMD 10B, which may be another example configuration of IMD 10 from FIGS. 1-3. IMD 10B of FIG. 4C may be configured substantially similarly to IMD 10A of FIG. 4A, with differences between them discussed herein.

IMD 10B may include a leadless, subcutaneously-implantable monitoring device, e.g. an ICM. IMD 10B includes housing having a base 40 and an insulative cover 42. Proximal electrode 16C and distal electrode 16D may be formed or placed on an outer surface of cover 42. Various circuitries and components of IMD 10B, e.g., described below with respect to FIG. 3, may be formed or placed on an inner surface of cover 42, or within base 40. In some examples, a battery or other power source of IMD 10B may be included within base 40. In the illustrated example, antenna 30B is formed or placed on the outer surface of cover 42, but may be formed or placed on the inner surface in some examples. In some examples, insulative cover 42 may be positioned over an open base 40 such that base 40 and cover 42 enclose the circuitries and other components and protect them from fluids such as body fluids.

Circuitries and components may be formed on the inner side of insulative cover 42, such as by using flip-chip technology. Insulative cover 42 may be flipped onto a base 40. When flipped and placed onto base 40, the components of IMD 10B formed on the inner side of insulative cover 42 may be positioned in a gap 44 defined by base 40. Electrodes 16C and 16D and antenna 30B may be electrically connected to circuitry formed on the inner side of insulative cover 42 through one or more vias (not shown) formed through insulative cover 42. Insulative cover 42 may be formed of sapphire (i.e., corundum), glass, parylene, and/or any other suitable insulating material. Base 40 may be formed from titanium or any other suitable material (e.g., a biocompatible material). Electrodes 16C and 16D may be formed from any of stainless steel, titanium, platinum, iridium, or alloys thereof. In addition, electrodes 16C and 16D may be coated with a material such as titanium nitride or fractal titanium nitride, although other suitable materials and coatings for such electrodes may be used.

In the example shown in FIG. 4C, the housing of IMD 10B defines a length L, a width W and thickness or depth D and is in the form of an elongated rectangular prism wherein the length L is much larger than the width W, which in turn is larger than the depth D, similar to IMD 10A of FIG. 4C. For example, the spacing between proximal electrode 64 and distal electrode 66 may range from 30 millimeters (mm) to 50 mm, from 35 mm to 45 mm, or be approximately 40 mm. In addition, IMD 10B may have a length L that ranges from 30 mm to about 70 mm. In other examples, the length L may range from 5 mm to 60 mm, 40 mm to 60 mm, 45 mm to 55 mm, or be approximately 45 mm. In addition, the width W may range from 3 mm to 15 mm, such as approximately 8 mm. The thickness of depth D of IMD 10B may range from 2 mm to 15 mm, from 3 to 5 mm, or be approximately 4 mm. IMD 10B may have a volume of three cubic centimeters (cm) or less, or 1.5 cubic cm or less, such as approximately 1.4 cubic cm.

In the example shown in FIG. 4C, once inserted subcutaneously within the patient, outer surface of cover 42 faces outward, toward the skin of the patient. In addition, as shown in FIG. 4C, proximal end 46 and distal end 48 are rounded to reduce discomfort and irritation to surrounding tissue once inserted

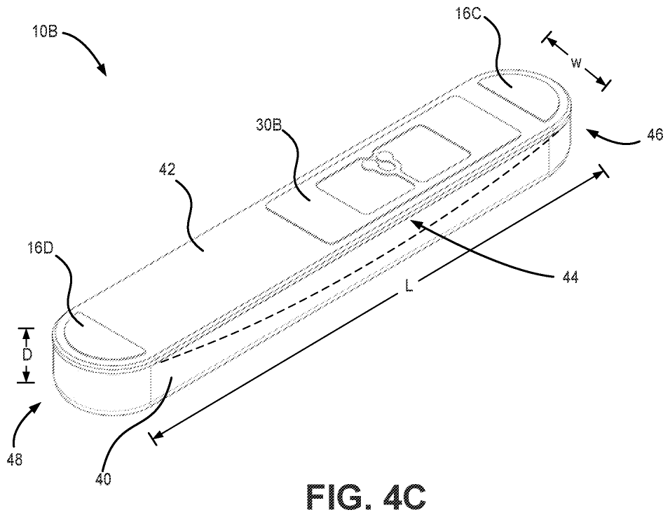

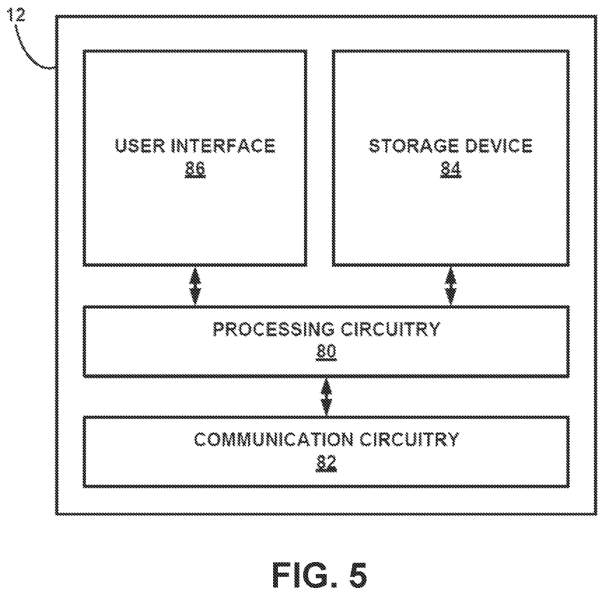

FIG. 5 is a block diagram illustrating an example configuration of components of external device 12. In the example of FIG. 5, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device. Communication circuitry 82 may be configured to transmit or receive signals via inductive coupling, electromagnetic coupling, Near Field Communication (NFC), Radio Frequency (RF) communication, Bluetooth, WiFi, or other proprietary or non-proprietary wireless communication schemes. Communication circuitry 82 may also be configured to communicate with devices other than IMD 10 via any of a variety of forms of wired and/or wireless communication and/or network protocols.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution.

Data exchanged between external device 12 and IMD 10 may include operational parameters. External device 12 may transmit data including computer readable instructions which, when implemented by IMD 10, may control IMD 10 to change one or more operational parameters and/or export collected data. For example, processing circuitry 80 may transmit an instruction to IMD 10 which requests IMD 10 to export collected data (e.g., PVC detection data and/or digitized cardiac EGMs) to external device 12. In turn, external device 12 may receive the collected data from IMD 10 and store the collected data in storage device 84. Processing circuitry 80 may implement any of the techniques described herein to analyze cardiac EGMs received from IMD 10, e.g., to determine whether a particular heartbeat is a PVC.

A user, such as a clinician or patient 4, may interact with external device 12 through user interface 86. User interface 86 includes a display (not shown), such as a liquid crystal display (LCD) or a light emitting diode (LED) display or other type of screen, with which processing circuitry 80 may present information related to IMD 10, e.g., cardiac EGMs, indications of detections of PVCs, and quantifications of detected PVCs, such as a quantification of PVC burden. In addition, user interface 86 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both.

FIG. 6 is a block diagram illustrating an example system that includes an access point 90, a network 92, external computing devices, such as a server 94, and one or more other computing devices 100A-100N (collectively, “computing devices 100”), which may be coupled to IMD 10 and external device 12 via network 92, in accordance with one or more techniques described herein. In this example, IMD 10 may use communication circuitry 54 to communicate with external device 12 via a first wireless connection, and to communicate with an access point 90 via a second wireless connection. In the example of FIG. 5, access point 90, external device 12, server 94, and computing devices 100 are interconnected and may communicate with each other through network 92.

Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with the patient. IMD 10 may be configured to transmit data, such as PVC detection information, PVC quantifications, and/or cardiac EGMs, to access point 90. Access point 90 may then communicate the retrieved data to server 94 via network 92.

In some cases, server 94 may be configured to provide a secure storage site for data that has been collected from IMD 10 and/or external device 12. In some cases, server 94 may assemble data in web pages or other documents for viewing by trained professionals, such as clinicians, via computing devices 100. One or more aspects of the illustrated system of FIG. 6 may be implemented with general network technology and functionality, which may be similar to that provided by the Medtronic CareLink® Network. In some examples, server 94 may communicate with computing device 100 via network 92. For example, server 94 may communicate an analysis of data, such as PVC detection information, to computing device 100, external device 12, or any other computing device via network 92. For example, server 94 may communicate that a particular heartbeat is a PVC beat to computing device 100, external device 12, or any other computing device via network 92.

In some examples, one or more of computing devices 100 may be a tablet or other smart device located with a clinician, by which the clinician may program, receive alerts from, and/or interrogate IMD 10. For example, the clinician may access data collected by IMD 10 through a computing device 100, such as when patient 4 is in in between clinician visits, to check on a status of a medical condition. In some examples, the clinician may enter instructions for a medical intervention for patient 4 into an application executed by computing device 100, such as based on a status of a patient condition determined by IMD 10, external device 12, server 94, or any combination thereof, or based on other patient data known to the clinician. Device 100 then may transmit the instructions for medical intervention to another of computing devices 100 located with patient 4 or a caregiver of patient 4. For example, such instructions for medical intervention may include an instruction to change a drug dosage, timing, or selection, to schedule a visit with the clinician, or to seek medical attention. In further examples, a computing device 100 may generate an alert to patient 4 based on a status of a medical condition of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

In the example illustrated by FIG. 6, server 94 includes a storage device 96, e.g., to store data retrieved from IMD 10, and processing circuitry 98. Although not illustrated in FIG. 5 computing devices 100 may similarly include a storage device and processing circuitry. Processing circuitry 98 may include one or more processors that are configured to implement functionality and/or process instructions for execution within server 94. For example, processing circuitry 98 may be capable of processing instructions stored in memory 96. Processing circuitry 98 may include or be coupled to communication circuitry that may include any suitable hardware, firmware, software or any combination thereof for communicating with another device. In some examples, a description of processing circuitry 98 outputting a signal, such as a classification, may include processing circuitry 98 causing communication circuitry of server 94 to output the signal. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98. Processing circuitry 98 of server 94 and/or the processing circuitry of computing devices 100 may implement any of the techniques described herein to analyze cardiac EGMs received from IMD 10, e.g., to determine whether a particular heartbeat is a PVC.

Storage device 96 may include a computer-readable storage medium or computer-readable storage device. In some examples, memory 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

Although the techniques for determining whether a particular heart beat is a PVC beat are described herein primarily (e.g., with respect to FIGS. 6-14) as being performed by processing circuitry 98 of server 94, such techniques may be performed, in whole or part, by processing circuitry of any one or more devices of system 2, such as processing circuitry 80 of external device 12, processing circuitry 50 of IMD 10, or processing circuitry of one or more computing devices 100.

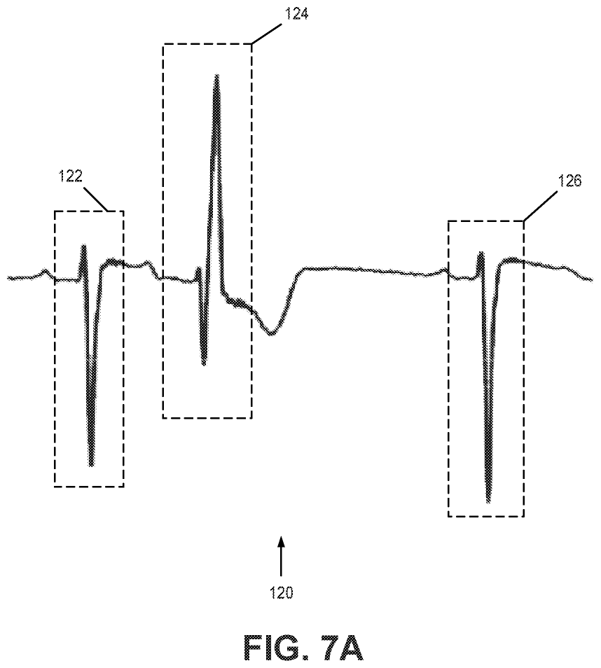

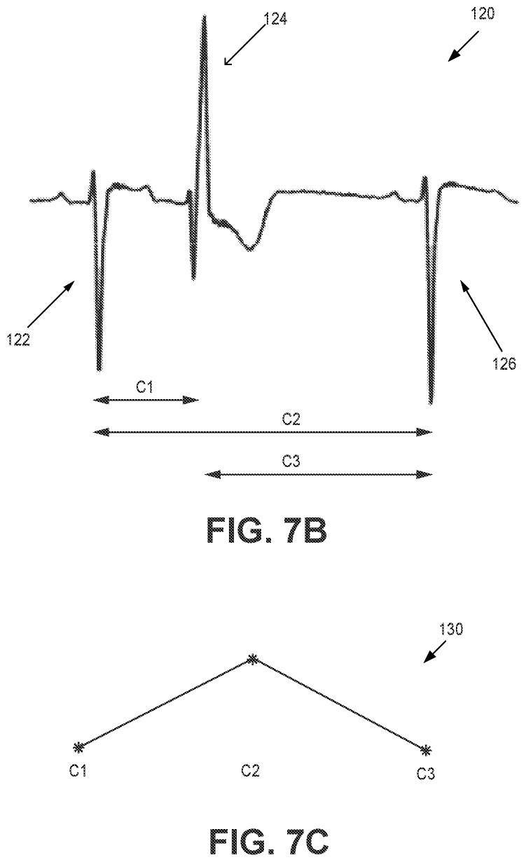

FIG. 7A is a graph illustrating an example of a sensed cardiac EGM signal 120 image including one or more particular heartbeats 124, a preceding adjacent heartbeat 122 immediately before the one or more particular heartbeats 124, and a subsequent adjacent heartbeat 126 immediately after the one or more particular heartbeats 124. Sensed cardiac EGM signal 120 may further include data on a heartbeat immediately before the preceding adjacent heartbeat 122 and may include data on a heartbeat immediately after the subsequent adjacent heartbeat 126. For example, this data may help in determining some RR intervals. In some examples, the sensed cardiac EGM signal 120 may be a window or segment beginning 75 samples before the R-wave peak of heartbeat 122 and ending at 95 samples after the R-wave peak of heartbeat 126. However, other amounts of sampling may be selected as well. While FIG. 7A illustrates an example of a sensed cardiac EGM signal having three consecutive heartbeats, in some examples, a sensed cardiac EGM signal may include more than three consecutive heartbeats, such as, but not limited to, four consecutive heartbeats or five consecutive heartbeats. In some examples, the one or more particular heartbeats 124 may include two consecutive heartbeats, such as for detecting a PVC couplet, or may include three consecutive heartbeats, such as for detecting a PVC triplet. In these examples, the preceding adjacent heartbeat 122 and the subsequent adjacent heartbeat 126 may be with respect to either the two consecutive heartbeats of the one or more particular heartbeats 124 that are in a PVC couplet or the three consecutive heartbeats of the one or more particular heartbeats 124 in a PVC triplet. While the following discussion is generally directed to the one or more particular heartbeats 124 being a particular heartbeat 124, it is not necessarily limited a particular heartbeat 124 being one heartbeat, and may, for example, be two consecutive heartbeats or three consecutive heartbeats, as discussed above.

For every beat, different features of the sensed cardiac EGM signal 120 may be extracted to help in differentiating between PVC and non-PVC beats which are then fed to the machine learning model for classification. In some examples, initially, a heartbeat, such as particular heartbeat 124, may be processed to determine if the heartbeat is noisy. If the heartbeat is determined to be noisy, then the machine learning model may be bypassed for the heartbeat and the heartbeat may automatically be classified as noise or a non-PVC beat.

Processing circuitry 98 may detect an R-wave peak of respective heartbeats in an EGM signal, such as the sensed cardiac EGM signal 120. To detect noise, processing circuitry 98 may determine if an R-wave of the heartbeat, such as particular heartbeat 124, is noisy. In some examples, to detect if the R-wave of the heartbeat is noisy, processing circuitry 98 may be configured to: 1) consider a number of samples, e.g., 25 samples, before and a number of samples, e.g., 25 samples, after the R-wave peak detected by IMD 10 during initial collection of the EGM signal; 2) subtract the mean from this segment of the EGM signal and compute the absolute value of the segment; 3) determine the sample with the maximum absolute value as the R-wave peak, which may differ from the R-wave peak identified by IMD 10 when initially collecting the EGM signal; 4) after determining the R-wave peak, consider a 32-sample window (15 samples before and 16 samples after) around the R-wave peak; 5) determine a first difference and second difference of this window of the signal; 6) for each sample, determine if there is a sign change in first difference value between the current and the next sample; 7) determine the number of such samples for which there is a sign change detected and the corresponding second difference value is greater or less than a threshold, e.g., greater than 40 or less than −40; 8) if the number of such samples is greater than a threshold number of samples, e.g., 8, then the R-wave is considered to be noisy.

If the R-wave is determined to be noisy, then processing circuitry 98 may label the current beat as a non-PVC beat and bypass the machine learning model for the beat. If the R-wave is determined to not be noisy, processing circuitry 98 may extract features from the sensed EGM signal 120 and feed sensed cardiac EGM signal 120 and different extracted features to the machine learning model for classification, as discussed below.

There are several characteristics of a cardiac EGM signal that may help distinguish a PVC from a normal heartbeat. For example, a coupling interval for a PVC heartbeat may be shorter than the coupling interval for a normal heartbeat. Accordingly, an RR interval between a PVC heartbeat and the preceding QRS wave may be shorter than for a normal heartbeat. In some examples, a compensatory pause may occur after a PVC beat, which leads to an RR interval between a PVC heartbeat and a subsequent heartbeat to be longer than an RR interval between a normal heartbeat and a subsequent heartbeat. In some examples, QRS and T-wave morphology may be different for a PVC heartbeat than for a normal heartbeat. In some examples, there may not be a P-wave preceding a QRS complex for a PVC heartbeat.

In some examples, one or more sensed cardiac EGM signals 120 may be received periodically. For example, a sensed cardiac EGM signal may be received daily, hourly, once every two hours, once every thirty minutes, or any other time period. In some examples, a clinician and/or user may request when a cardiac EGM signal is obtained.

FIG. 7B is a graph illustrating an example of a plurality of correlation coefficients being determined for the sensed cardiac EGM signal 120 image in FIG. 7A. As shown as an example in FIG. 7B, C1 represents the correlation coefficient between heartbeat 122 and heartbeat 124, C2 represents the correlation coefficient between heartbeat 122 and heartbeat 126, and C3 represents the correlation coefficient between heartbeat 124 and heartbeat 126. Processing circuitry 98 may perform feature extraction and extract the correlation coefficients, such as C1, C2, and C3, from the sensed cardiac EGM signal 120. In some examples, processing circuitry 98 may consider 25 samples before and 75 samples after each respective R-wave peak of heartbeats 122, 124, 126 in the sensed cardiac EGM signal in determining the correlation coefficients C1, C2, C3.

As shown as an example in FIG. 7C, processing circuitry 98 may convert the extracted correlation coefficients, such as C1, C2, and C3, to a feature image 130 to be applied to a machine learning model. In some examples, feature image 130 may be an array of numbers, such as a two-dimensional array of numbers or a one-dimensional array of numbers. In some examples, feature image 130 may be a digital image. For example, processing circuitry 98 may convert the extracted correlation coefficients, using a programming script, such as a MATLAB® script, to a respective digital image (e.g., example of feature image 130), such as a JPEG image, to be applied to a machine learning model. In some examples, when the particular heartbeat 124 is a PVC beat, values of correlation coefficients C1 and C3 may be small since the morphology of the particular heartbeat 124 will be different from the adjacent normal beats 122, 126 to the particular heartbeat 124. In addition, when the particular heartbeat 124 is a PVC beat, C2 may be large since the morphologies of the two normal beats 122, 126 will be similar.

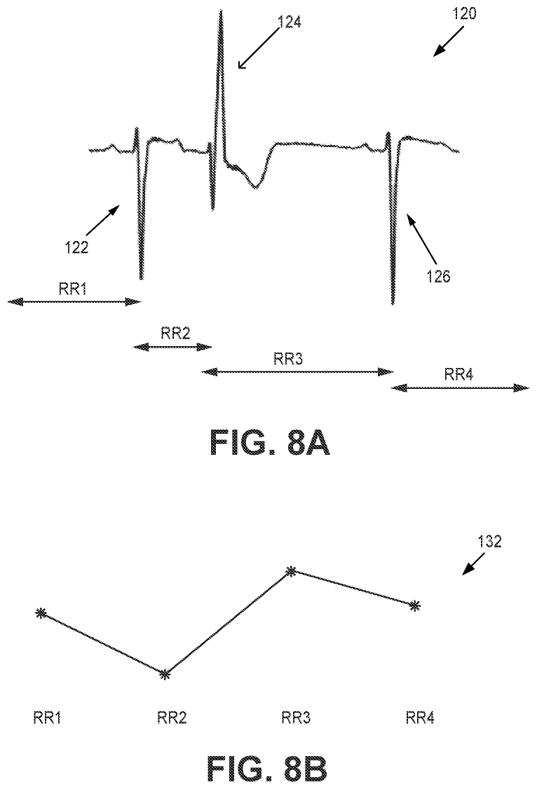

FIG. 8A is a graph illustrating an example of a plurality of RR intervals being determined for the sensed cardiac EGM signal 120 in FIG. 7A. As shown as an example in FIG. 8A, is a graph illustrating an example of four RR intervals RR1, RR2, RR3, RR4 of the sensed cardiac EGM 120. An RR interval is determined as the time interval between the maximum R-wave amplitude in a beat and a maximum R-wave amplitude of a neighboring beat. As an example, RR1 is the time interval between the maximum R-wave amplitude of heartbeat 122 and the maximum R-wave amplitude of a heartbeat preceding heartbeat 122. RR2 is the time interval between the maximum R-wave amplitude of heartbeat 122 and the maximum R-wave amplitude of heartbeat 124. RR3 is the time interval between the maximum R-wave amplitude of heartbeat 124 and the maximum R-wave amplitude of heartbeat 126. RR4 is the time interval between the maximum R-wave amplitude of heartbeat 126 and the maximum R-wave amplitude of a heartbeat immediately subsequent to heartbeat 126.

Processing circuitry 98 may perform feature extraction and extract the RR intervals, such as RR1, RR2, RR3, and RR4, from the sensed cardiac EGM signal 120. In some examples, processing circuitry 98 may consider 25 samples before and 75 samples after each respective R-wave peak of heartbeats in the sensed cardiac EGM signal in determining the RR intervals RR1, RR2, RR3, RR4.

As shown as an example in FIG. 8B, processing circuitry 98 may convert the extracted RR intervals, such as RR1, RR2, RR3, and RR4, to a feature image 132 to be applied to a machine learning model. In some examples, when the particular heartbeat 124 is a PVC beat, RR2 may be the shortest RR interval and RR3 may be the longest RR interval since there is generally a compensatory pause after a PVC beat.

Processing circuitry 98 may perform feature extraction and extract one or more of maximum QRS amplitude 141, minimum QRS amplitude 142, difference between maximum QRS amplitude and minimum QRS amplitude 143, number of samples between maximum QRS amplitude and minimum QRS amplitude 144, maximum slope of the QRS wave 145, minimum slope of the QRS wave 146, difference of slope value between the maximum slope of the QRS wave and the minimum slope of the QRS wave 147, and number of samples between the maximum slope of the QRS wave and the minimum slope of the QRS wave 148 from the sensed cardiac EGM signal 120.

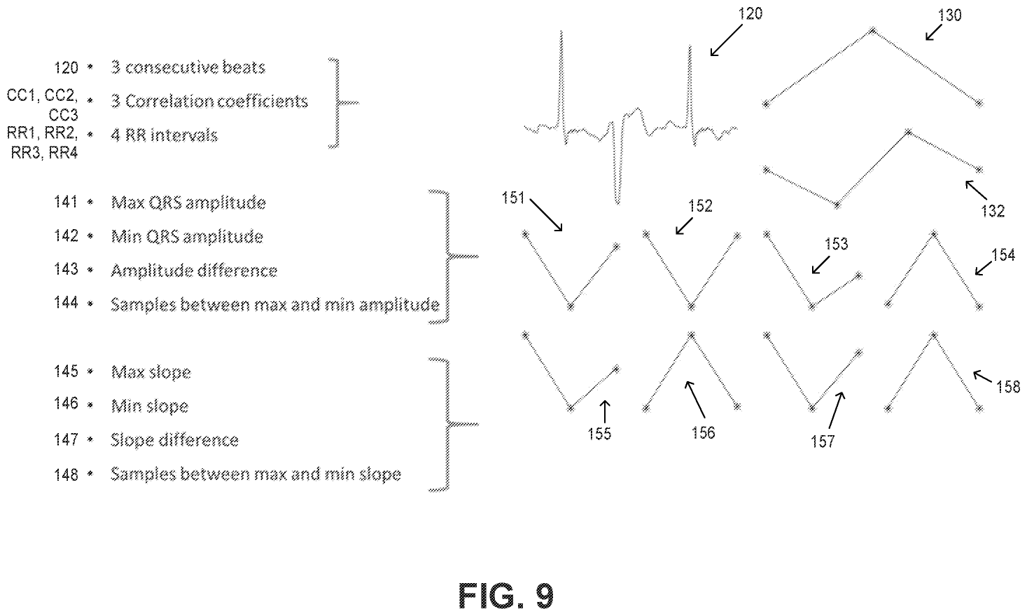

As shown as an example in FIG. 9, processing circuitry 98 may convert one or more of the extracted values discussed above 141, 142, 143, 144, 145, 146, 147, 148 to a respective feature image 151, 152, 153, 154, 155, 156, 157, 158 to be applied to a machine learning model.

As shown as an example in FIG. 9, processing circuitry 98 may weight one or more of the sensed cardiac EGM signal 120 image, feature correlation coefficient image 130, feature RR interval image 132, feature maximum QRS amplitude image 151, feature minimum QRS amplitude image 152, feature difference between maximum QRS amplitude and minimum QRS amplitude image 153, feature number of samples between maximum QRS amplitude and minimum QRS amplitude image 154, feature maximum slope of the QRS wave image 155, feature minimum slope of the QRS wave image 156, feature difference of slope value between the maximum slope of the QRS wave and the minimum slope of the QRS wave image 157, or feature number of samples between the maximum slope of the QRS wave and the minimum slope of the QRS wave image 158. In some examples, weighing images includes adjusting the respective sizes of images to be applied to the machine learning model. For example, if the sensed cardiac EGM signal 120 image has a weight of four, feature correlation coefficient image 130 has a weight of two, and feature maximum QRS amplitude image 151 has a weight of one, a size of the sensed cardiac EGM signal 120 image will be four times larger than the feature maximum QRS amplitude image 151 and twice as large as the feature correlation coefficient image 130.

In some examples, a machine learning model may give more weight to larger images than to smaller images. Processing circuitry 98 may provide greater weight to extracted features, which lead to larger feature images of the respective extracted features, that have a greater effect on determining whether a particular heartbeat 124 is a PVC.

In some examples, processing circuitry 98 may give the sensed cardiac EGM signal 120 image the highest weight. For example, in FIG. 9, the sensed cardiac EGM signal 120 image is given a weight of four. In other examples, processing circuitry 98 may give different values of the weight. Processing circuitry 98 may give one or more of the feature correlation coefficient image 130 or the feature RR interval image 132 the next highest weight behind the sensed cardiac EGM signal 120 image. For example, in FIG. 9, the feature correlation coefficient image 130 or the feature RR interval image 132 are each given a weight of 2. In other examples, processing circuitry 98 may give different values of the weight.

As an example, the sensed cardiac EGM signal 120 image, the feature correlation coefficient image 130, and the feature RR interval image 132 may have the greatest effect of the feature images of the extracted features on determining whether a particular heartbeat 124 is a PVC. Accordingly, processing circuitry 98 may apply the greatest weights to the sensed cardiac EGM signal 120 image, the feature correlation coefficient image 130, and the feature RR interval image 132

Processing circuitry 98 may give one or more of the feature maximum QRS amplitude image 151, feature minimum QRS amplitude image 152, feature difference between maximum QRS amplitude and minimum QRS amplitude image 153, feature number of samples between maximum QRS amplitude and minimum QRS amplitude image 154, feature maximum slope of the QRS wave image 155, feature minimum slope of the QRS wave image 156, feature difference of slope value between the maximum slope of the QRS wave and the minimum slope of the QRS wave image 157, or feature number of samples between the maximum slope of the QRS wave and the minimum slope of the QRS wave image 158 the lowest weight. For example, in FIG. 9, the feature images 151, 152, 153, 154, 155, 156, 157, 158 are each given a weight of one. In other examples, processing circuitry 98 may give different values of the weight.

For example, in FIG. 9, the image of the sensed cardiac EGM signal 120 is twice as large as each of the feature correlation coefficient image 130 and the feature RR interval image 132 due the weights applied by processing circuitry 98. In addition, the image of the sensed cardiac EGM signal 120 is four times as large as each of the feature images 151, 152, 153, 154, 155, 156, 157, 158 due the weights applied by processing circuitry 98.

Processing circuitry 98 may apply one of more of the weighted images 120, 130, 132, 151, 152, 153, 154, 155, 156, 157, 158 to a machine learning model to determine whether a particular heartbeat in the sensed cardiac electrogram is a PVC beat. In response to determining that the particular heartbeat is a PVC beat, processing circuitry 98 may cause a classification that the particular heartbeat is a PVC beat to be output. For example, the classification may be output to a clinician computing device.

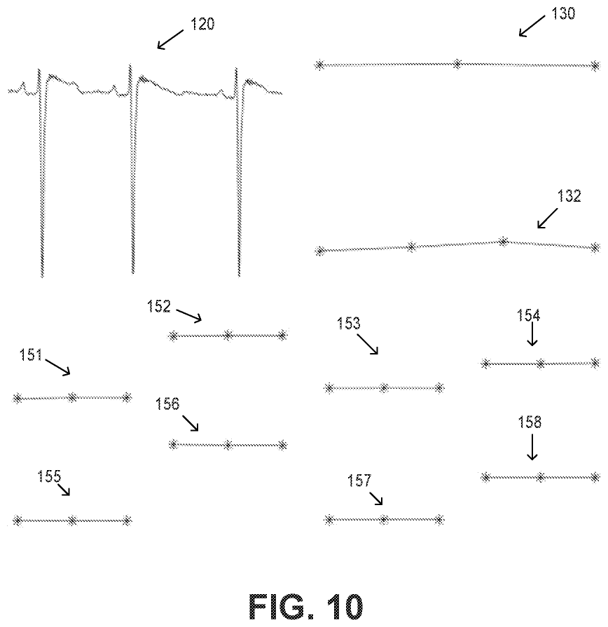

FIG. 10 shows an example of weighted images of the sensed cardiac EGM signal 120 image, feature correlation coefficient image 130, feature RR interval image 132, feature maximum QRS amplitude image 151, feature minimum QRS amplitude image 152, feature difference between maximum QRS amplitude and minimum QRS amplitude image 153, feature number of samples between maximum QRS amplitude and minimum QRS amplitude image 154, feature maximum slope of the QRS wave image 155, feature minimum slope of the QRS wave image 156, feature difference of slope value between the maximum slope of the QRS wave and the minimum slope of the QRS wave image 157, or feature number of samples between the maximum slope of the QRS wave and the minimum slope of the QRS wave image 158 for a normal heartbeat.

In some examples, processing circuitry 98 may normalize axes of the sensed cardiac EGM signal 120 image and one or more of the feature images 130, 132, 151, 152, 153, 154, 155, 156, 157, 158 to be applied to the machine learning model. Processing circuitry 98 may normalize axes of the weighted sensed cardiac EGM signal 120 image and one or more of the weighted feature images 130, 132, 151, 152, 153, 154, 155, 156, 157, 158 to be applied to the machine learning model. In some examples in which processing circuitry 98 normalizes the axes of the images, processing circuitry 98 may weight cardiac EGM signal 120 image and one or more of images 130, 132, 151, 152, 153, 154, 155, 156, 157, 158 before or after processing circuitry 98 normalizes the axes.

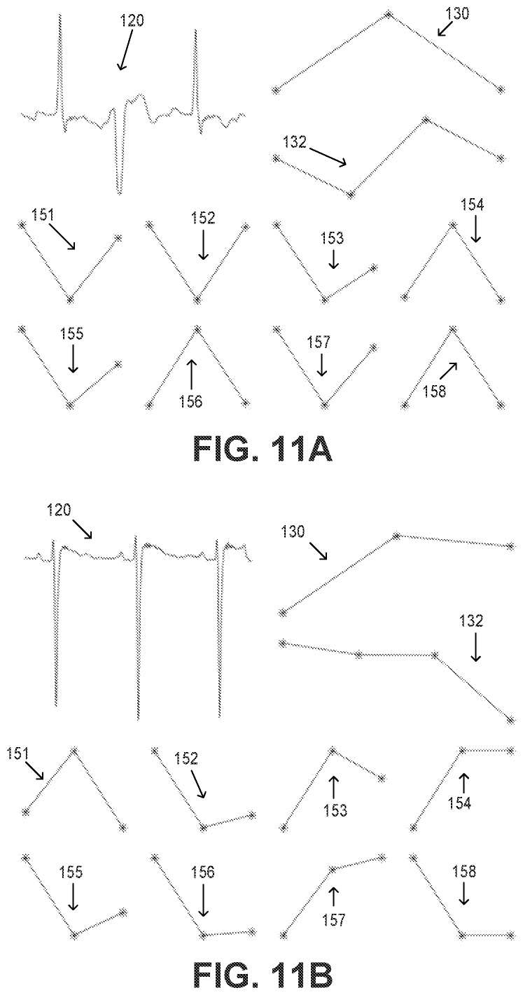

FIGS. 11A and 11B show examples of weighted images of the sensed cardiac EGM signal 120 image, feature correlation coefficient image 130, feature RR interval image 132, feature maximum QRS amplitude image 151, feature minimum QRS amplitude image 152, feature difference between maximum QRS amplitude and minimum QRS amplitude image 153, feature number of samples between maximum QRS amplitude and minimum QRS amplitude image 154, feature maximum slope of the QRS wave image 155, feature minimum slope of the QRS wave image 156, feature difference of slope value between the maximum slope of the QRS wave and the minimum slope of the QRS wave image 157, or feature number of samples between the maximum slope of the QRS wave and the minimum slope of the QRS wave image 158 that did not have the axes normalized.

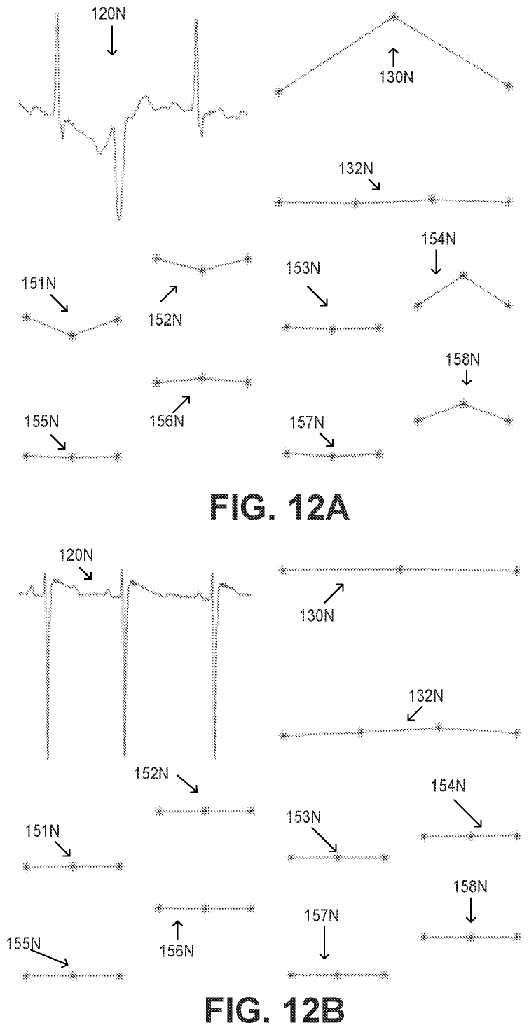

FIGS. 12A and 12B show examples of processing circuitry 98 normalizing the axes of weighted images of the sensed cardiac EGM signal 120N image, feature correlation coefficient image 130N, feature RR interval image 132N, feature maximum QRS amplitude image 151N, feature minimum QRS amplitude image 152N, feature difference between maximum QRS amplitude and minimum QRS amplitude image 153N, feature number of samples between maximum QRS amplitude and minimum QRS amplitude image 154N, feature maximum slope of the QRS wave image 155N, feature minimum slope of the QRS wave image 156N, feature difference of slope value between the maximum slope of the QRS wave and the minimum slope of the QRS wave image 157N, or feature number of samples between the maximum slope of the QRS wave and the minimum slope of the QRS wave image 158N to be applied to the machine learning model.

In some examples, processing circuitry 98 may normalize one or more axes, such as a y-axis, of the converted images and/or the weighted images based on the minimum and maximum value of each respective feature that was determined from the development dataset. In some examples, processing circuitry 98 may normalize one or more axes of the converted images and/or the weighted images to be between 0 and 1 based on minimum and maximum values in the dataset of the images or by dividing each data point of the dataset of the respective images by a constant, such as the standard deviation of the dataset of the respective images, maximum value of the dataset of the respective images, minimum value of the dataset of the respective images, mean of the dataset of the respective images, median of the dataset of the respective images, etc.

In some examples, by extracting particular features from a cardiac EGM that have been determined according to the inventive techniques of this disclosure to correspond to PVCs, as discussed above, to be applied to a machine learning model, the machine learning model may be more focused and spend a majority of its nodes on determining whether a particular heartbeat is a PVC, which may lead to more accurate results.

In some examples, by extracting one or more particular features from sensed cardiac EGM signal 120, and converting the extracted features into images, and applying the EGM signal 120 image and feature images to a machine learning model to determine whether a particular heartbeat 124 is a PVC, processing circuitry 98 may be able to determine whether a particular heartbeat 124 is a PVC with great accuracy, specificity, and sensitivity. In addition, in some examples, by weighting and/or normalizing the EGM signal 120 image and feature images and applying the weighted and/or normalized EGM signal 120 image and feature images to a machine learning model to determine whether a particular heartbeat 124 is a PVC, processing circuitry 98 may be able to determine whether a particular heartbeat 124 is a PVC with great accuracy, specificity, and sensitivity. This may facilitate determinations of PVC burden, cardiac wellness, and risk of sudden cardiac death, and may lead to clinical interventions to suppress PVCs such as medications and PVC ablations.

Processing circuitry 98 may be configured to execute an artificial intelligence (AI) engine that operates according to one or more models, such as machine learning models. Machine learning models may include any number of different types of machine learning models, such as neural networks, deep neural networks, convolution neural networks, recurrent neural networks, such as long short term memory networks, dense neural networks, and the like. In some examples, various feature inputs to the AI engine may be fed as direct inputs to different layers in a network and not necessarily prior to the convolution layers. Although described with respect to machine learning models, the techniques described in this disclosure are also applicable to other types of AI models, including rule-based models, finite state machines, and the like.

Machine learning may generally enable a computing device to analyze input data and identify an action to be performed responsive to the input data. Each machine learning model may be trained using training data that reflects likely input data. The training data may be labeled or unlabeled (meaning that the correct action to be taken based on a sample of training data is explicitly stated or not explicitly stated, respectively).

The training of the machine learning model may be guided (in that a designer, such as a computer programmer, may direct the training to guide the machine learning model to identify the correct action in view of the input data) or unguided (in that the machine learning model is not guided by a designer to identify the correct action in view of the input data). In some instances, the machine learning model is trained through a combination of labeled and unlabeled training data, a combination of guided and unguided training, or possibly combinations thereof. Examples of machine learning include nearest neighbor, naïve Bayes, decision trees, linear regression, support vector machines, neural networks, k-Means clustering, Q-learning, temporal difference, deep adversarial networks, evolutionary algorithms or other supervised, unsupervised, semi-supervised, or reinforcement learning algorithms to train one or more models.

Processing circuitry 98 may utilize machine learning, such as a deep learning algorithm or model (e.g., a neural network or deep belief network), to generate a score indicative of whether a particular heartbeat 124 may be a PVC to determine whether a particular heartbeat 124 is a PVC. Processing circuitry 98 may train a deep learning model to represent a relationship of the images discussed above to whether a particular heartbeat is a PVC. For example, processing circuitry 98 may train the deep learning model using images from EGM signals from other patients. In some examples, processing circuitry 98 may train the deep leaning model by adjusting the weights of a hidden layer of a neural network model to balance the contribution of each input (e.g., characteristics of the images input) according to determining whether a heartbeat is a PVC.

Once the deep learning model is trained, processing circuitry 98 may obtain and apply data, such as the EGM signal 120 image and feature images of features extracted from EGM signal 120, to the trained deep learning model. For example, the input images may include normalized weighted images of one or more of the sensed cardiac EGM signal 120N image, feature correlation coefficient image 130N, feature RR interval image 132N, feature maximum QRS amplitude image 151N, feature minimum QRS amplitude image 152N, feature difference between maximum QRS amplitude and minimum QRS amplitude image 153N, feature number of samples between maximum QRS amplitude and minimum QRS amplitude image 154N, feature maximum slope of the QRS wave image 155N, feature minimum slope of the QRS wave image 156N, feature difference of slope value between the maximum slope of the QRS wave and the minimum slope of the QRS wave image 157N, or feature number of samples between the maximum slope of the QRS wave and the minimum slope of the QRS wave image 158N.

The output of the deep learning model may include a score that indicates whether or not the particular heartbeat is a PVC. For example, the score may be a probability that the particular heartbeat is a PVC. Processing circuitry 98 may cause the score to be displayed to a clinician to aid in determining whether or not the patient should receive treatment based on the having PVC(s). In some examples, the output of the deep learning model may include a classification of a type of PVC beat, such as monomorphic, polymorphic, bigeminy, trigeminy, and interpolated PVCs. In some examples, processing circuitry 98 may determine a classification of a type of PVC beat, such as monomorphic, polymorphic, bigeminy, trigeminy, and interpolated PVCs, based on the determination of whether or not the particular heartbeat is a PVC and/or a score that indicates whether or not the particular heartbeat is a PVC for a sequence of heartbeats. For example, processing circuitry 98 may determine patient 4 has trigeminy if every third beat in a sequence of heartbeats is determined to be a PVC. For example, the sequence of heartbeats may include 5, 10, 15, 20, or 50 heartbeats. However, other amounts of heartbeats, such as greater than 50 heartbeats or less than 50 heartbeats, may also be used for the sequence of heartbeats. In some examples, the deep learning model may also be trained to determine a location of the origin of a PVC, such as whether the detected PVC beat is originating from the septum, left ventricle, right ventricle, right ventricle outflow tract, etc. Processing circuitry 98 may obtain and apply data, such as the EGM signal 120 image and feature images of features extracted from EGM signal 120, to the trained deep learning model, to determine whether a particular heartbeat is a PVC beat and/or determine a location of the origin of the detected PVC beat, such as whether the detected PVC beat is originating from the septum, left ventricle, right ventricle, right ventricle outflow tract.

In some examples, when processing circuitry 98 determines a particular heartbeat is a PVC beat, a score and/or correlation coefficients of the PVC beat may be stored and may be compared with the scores and correlation coefficients of the previously detected PVC beats to determine if they are monomorphic or polymorphic PVC beats. In some examples, when processing circuitry 98 determines the scores and/or and correlation coefficients of all the PVC beats are similar, then processing circuitry 98 may determine the PVC beats may be monomorphic PVC beats that exhibit the same morphologies. In some examples, when processing circuitry 98 determines the scores and correlation coefficients have a difference greater than a difference threshold, processing circuitry 98 determines the PVC beats may be polymorphic PVCs. In some examples, processing circuitry 98 may determine, based on the scores and/or correlation coefficients, how many different morphologies of PVC beats are present in a patient.

For example, a ventricular bigeminy pattern is when each normal beat is followed by a PVC beat. When processing circuitry 98 detects a PVC pattern of: Normal beat-PVC beat-Normal beat-PVC beat-Normal beat-PVC beat, then processing circuitry 98 determines that this PVC pattern is a bigeminy pattern and may provide a corresponding alert. Ventricular trigeminy pattern is when a PVC beat occurs every third beat. When processing circuitry 98 detects a PVC pattern of: Normal beat-Normal beat-PVC beat-Normal beat-Normal beat-PVC beat-Normal beat-Normal beat-PVC beat, then processing circuitry 98 determines that this PVC pattern is a trigeminy pattern and may provide a corresponding alert. When processing circuitry 98 detects a PVC beat occurs every fourth beat, then processing circuitry 98 may determine this PVC pattern as a quadrigeminy pattern. For example, a quadrigeminy pattern will look like: Normal beat-Normal beat-Normal beat-PVC beat-Normal beat-Normal beat-Normal beat-PVC beat-Normal beat-Normal beat-Normal beat-PVC beat.

A detected PVC can be classified as an interpolated PVC based on the RR interval patterns. If the detected PVC does not have a compensatory pause and P-P interval for normal beats remain approximately constant, then these PVCs can be classified as interpolated PVCs by the AI.

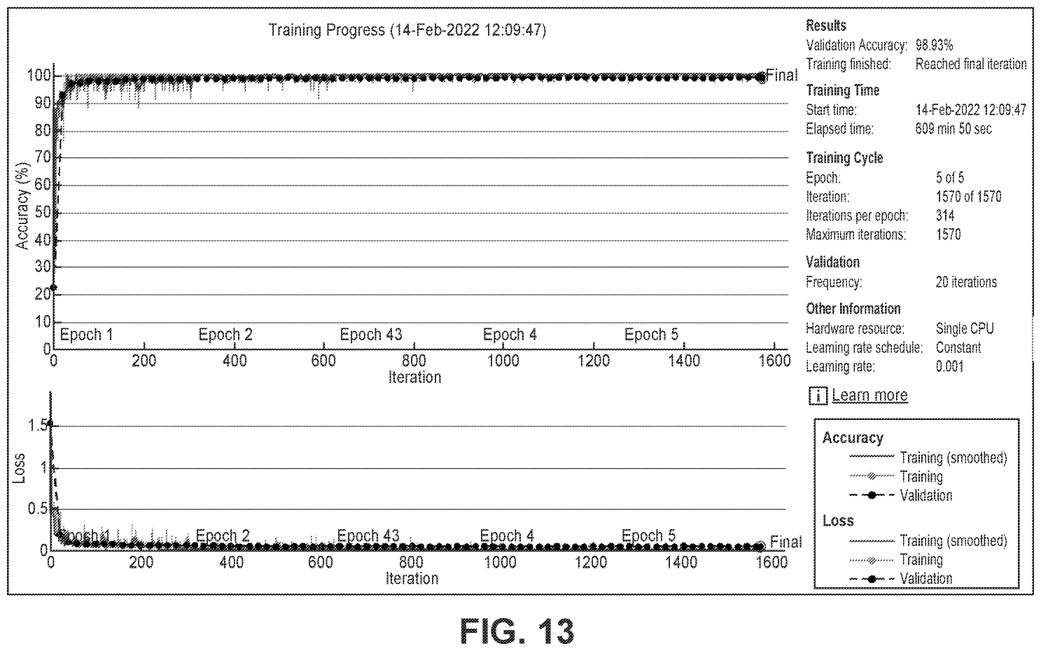

FIG. 13 shows an example of processing circuitry 98 applying the normalize weighted images of the sensed cardiac EGM signal 120N image, feature correlation coefficient image 130N, feature RR interval image 132N, feature maximum QRS amplitude image 151N, feature minimum QRS amplitude image 152N, feature difference between maximum QRS amplitude and minimum QRS amplitude image 153N, feature number of samples between maximum QRS amplitude and minimum QRS amplitude image 154N, feature maximum slope of the QRS wave image 155N, feature minimum slope of the QRS wave image 156N, feature difference of slope value between the maximum slope of the QRS wave and the minimum slope of the QRS wave image 157N, or feature number of samples between the maximum slope of the QRS wave and the minimum slope of the QRS wave image 158N, as shown in FIGS. 12A and 12B, to a machine learning model. As shown in the example of FIG. 13, the machine learning model resulted an accuracy of 98.93%.

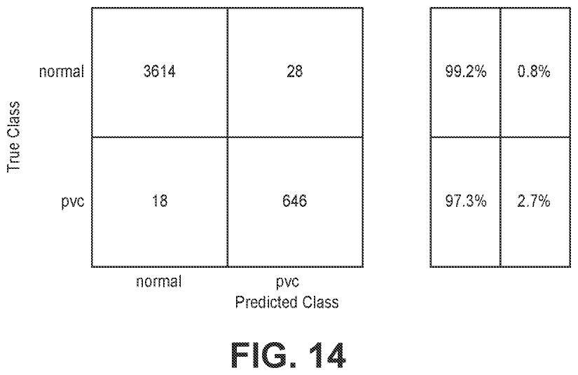

FIG. 14 shows an example of the confusion matrix on the validation dataset of FIG. 13. As shown in the example of FIG. 14, the sensitivity of the PVC detection was found to be 97.3% and the specificity was found to be 99.2%.

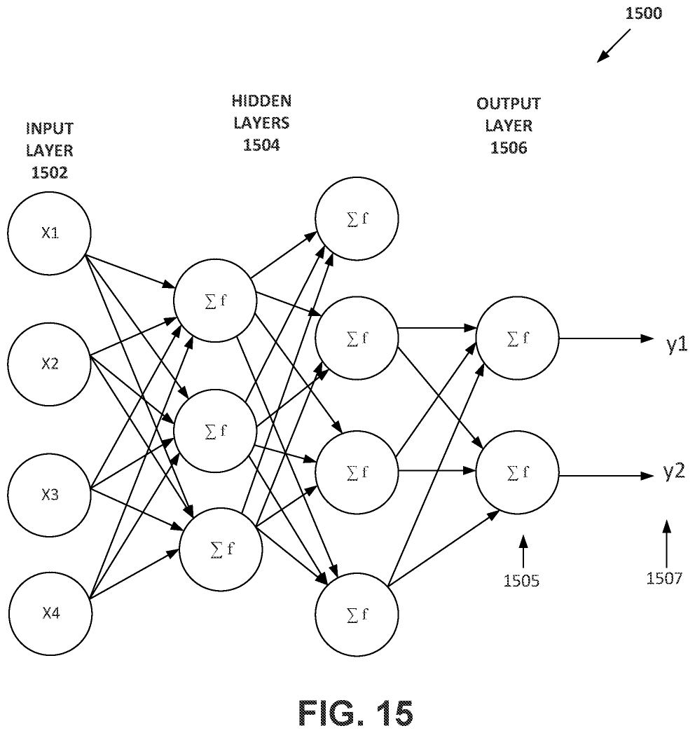

FIG. 15 is a conceptual diagram illustrating an example machine learning model 1500 configured to determine whether a particular heartbeat in the sensed cardiac electrogram is a PVC beat. Machine learning model 1500 is an example of the machine learning model discussed above. Machine learning model 1500 is an example of a deep learning model, or deep learning algorithm, trained to determine whether a particular heartbeat in the sensed cardiac electrogram is a PVC beat and/or a score indicative of whether a particular heartbeat may be a PVC beat. One or more of IMD 10, external device 12, server 94, and/or computing device(s) 100 may train, store, and/or utilize machine learning model 1500, but other devices may apply inputs associated with a particular patient to machine learning model 1500 in other examples. As discussed above, other types of machine learning and deep learning models or algorithms may be utilized in other examples. For examples, a convolutional neural network model of ResNet-18 may be used. Some non-limiting examples of models that may be used for transfer learning include AlexNet, VGGNet, GoogleNet, ResNet50, or DenseNet, etc. Some non-limiting examples of machine learning techniques include Support Vector Machines, K-Nearest Neighbor algorithm, and Multi-layer Perceptron.

As shown in the example of FIG. 15, machine learning model 1500 may include three layers. These three layers include input layer 1502, hidden layer 1504, and output layer 1506. Output layer 1506 comprises the output from the transfer function 1505 of output layer 1506. Input layer 1502 represents each of the input values X1 through X4 provided to machine learning model 1500. In some examples, the input values may any of the of values input into the machine learning model, as described above. For example, the input values may be one of more of the weighted images 120, 130, 132, 151, 152, 153, 154, 155, 156, 157, 158, as described above. In some examples, the input values may be one or more of the normalized weighted images of the sensed cardiac EGM signal 120N image, feature correlation coefficient image 130N, feature RR interval image 132N, feature maximum QRS amplitude image 151N, feature minimum QRS amplitude image 152N, feature difference between maximum QRS amplitude and minimum QRS amplitude image 153N, feature number of samples between maximum QRS amplitude and minimum QRS amplitude image 154N, feature maximum slope of the QRS wave image 155N, feature minimum slope of the QRS wave image 156N, feature difference of slope value between the maximum slope of the QRS wave and the minimum slope of the QRS wave image 157N, or feature number of samples between the maximum slope of the QRS wave and the minimum slope of the QRS wave image 158N. In addition, in some examples input values of machine learning model 1500 may include additional data, such as data relating to one or more additional parameters of patient 4.

Each of the input values for each node in the input layer 1502 is provided to each node of hidden layer 1504. In the example of FIG. 15, hidden layers 1504 include two layers, one layer having four nodes and the other layer having three nodes, but fewer or greater number of nodes may be used in other examples. Each input from input layer 1502 is multiplied by a weight and then summed at each node of hidden layers 1504. During training of machine learning model 1500, the weights for each input are adjusted to establish the relationship between the cardiac electrogram to determining whether a particular heartbeat in the sensed cardiac electrogram is a PVC beat and/or determining a score indicative of whether a particular heartbeat may be a PVC. In some examples, one hidden layer may be incorporated into machine learning model 1500, or three or more hidden layers may be incorporated into machine learning model 1500, where each layer includes the same or different number of nodes.

The result of each node within hidden layers 1504 is applied to the transfer function of output layer 1506. The transfer function may be liner or non-linear, depending on the number of layers within machine learning model 1500. Example non-linear transfer functions may be a sigmoid function or a rectifier function. The output 1507 of the transfer function may be a classification that the particular heartbeat is a PVC beat and/or a score indicative of whether a particular heartbeat may be a PVC beat that is generated by a computing device or computing system, such as by processing circuitry 98, in response to applying one of more of the weighted images 120, 130, 132, 151, 152, 153, 154, 155, 156, 157, 158 to machine learning model 1500.

As shown in the example above, by extracting one or more particular features from sensed cardiac EGM signal 120, and converting the extracted features into images, and applying the EGM signal 120 image and feature images to a machine learning model, such as machine learning model 1500, to determine whether a particular heartbeat 124 is a PVC, processing circuitry 98 is able to determine whether a particular heartbeat 124 is a PVC with great accuracy, specificity, and sensitivity. In addition, in some examples, by weighting and/or normalizing the EGM signal 120 image and feature images and applying the weighted and/or normalized EGM signal 120 image and feature images to a machine learning model to determine whether a particular heartbeat 124 is a PVC, processing circuitry 98 is able to determine whether a particular heartbeat 124 is a PVC with great accuracy, specificity, and sensitivity. This may facilitate determinations of PVC burden, cardiac wellness, and risk of sudden cardiac death, and may lead to clinical interventions to suppress PVCs such as medications and PVC ablations.

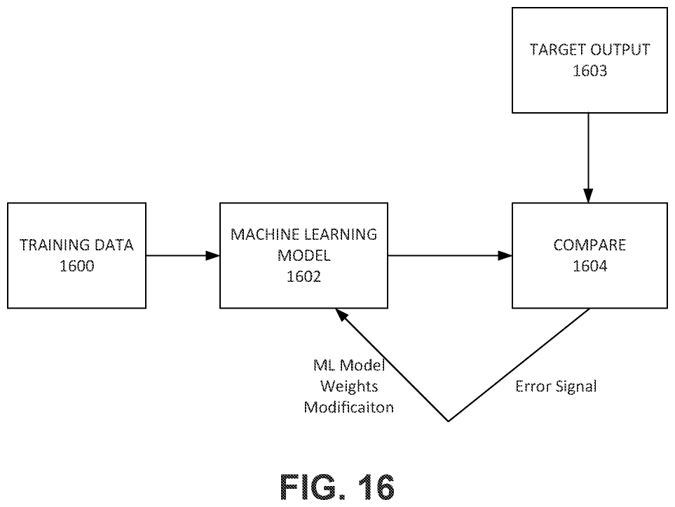

FIG. 16 is an example of the machine learning model 1602 being trained using supervised and/or reinforcement learning techniques. The machine learning model 1602 may be implemented using any number of models for supervised and/or reinforcement learning, such as but not limited to, an artificial neural network, a decision tree, naïve Bayes network, support vector machine, or k-nearest neighbor model, to name only a few examples. In some examples, one or more of IMD 10, external device 12, server 94, and/or computing device(s) 100 initially trains the machine learning model 1602 based on a training set of metrics and corresponding to a PVC beat. The training set 1600 may include a set of feature vectors, where each feature in the feature vector represents a value for a particular metric. One or more of IMD 10, external device 12, server 94, and/or computing device(s) 100 may select a training set comprising a set of training instances, each training instance comprising an association between one or more respective cardiac electrogram features of a respective feature image and a respective PVC beat. A prediction or classification by the machine learning model 1602 may be compared 1604 to the target output 1603, and an error signal and/or machine learning model weights modification may sent/applied to the machine learning model 1602 based on the comparison to modify/update the machine learning model 1602. For example, one or more of IMD 10, external device 12, server 94, and/or computing device(s) 100 may, for each training instance in the training set, modify, based on the respective cardiac electrogram features and the respective PVC beat of the training instance, the machine learning model 1602 to change a score generated by the machine learning model 1602 in response to subsequent PVC beats applied to the machine learning model 1602.

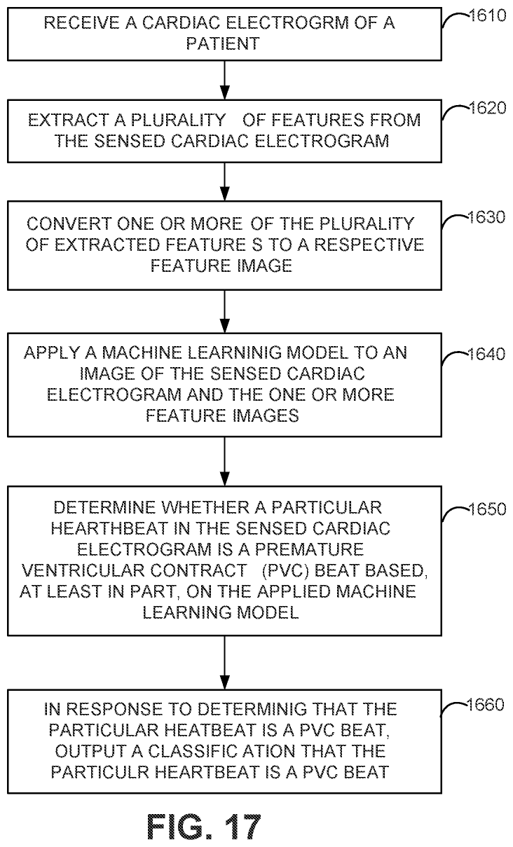

FIG. 17 is a flow diagram illustrating an example technique for medical system 2. As indicated by FIG. 17, processing circuitry 98 may receive a sensed cardiac electrogram of a patient (1610). Processing circuitry 98 may extract a plurality of features from the sensed cardiac electrogram (1620). Processing circuitry 98 may convert one or more of the plurality of extracted features to a respective feature image (1630). Processing circuitry 98 may apply a machine learning model, such as machine learning model 1500, to an image of the sensed cardiac electrogram and the one or more feature images (1640). The machine learning model may be trained using cardiac electrogram data for a plurality of patients. Processing circuitry 98 may determine whether a particular heartbeat in the sensed cardiac electrogram is a PVC beat based, at least in part, on the applied machine learning model (1650). In response to processing circuitry 98 determining that the particular heartbeat is a PVC beat, processing circuitry 98 may output a classification that the particular heartbeat is a PVC beat (1660).

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

Various aspects of the techniques may enable the following examples.

Example 1: A medical system includes a plurality of electrodes configured to sense a cardiac electrogram of a patient; and processing circuitry configured to: perform feature extraction of the sensed cardiac electrogram to extract a plurality of features from the sensed cardiac electrogram; convert one or more of the plurality of extracted features to a respective feature image; apply a machine learning model, trained using cardiac electrogram data for a plurality of patients, to an image of the sensed cardiac electrogram and at least the respective feature image to determine whether one or more particular heartbeats in the sensed cardiac electrogram indicate a premature ventricular contraction (PVC) beat; and in response to the determination that the one or more particular heartbeats indicates a PVC beat, output a classification that the one or more particular heartbeats is a PVC beat.

Example 2: The medical system of example 1, wherein the sensed cardiac electrogram comprises signals of consecutive heartbeats, the signals of the consecutive heartbeats including a signal of the one or more particular heartbeats, a signal of a heartbeat immediately preceding the one or more particular heartbeats and a signal of a heartbeat immediately after the one or more particular heartbeats.

Example 3: The medical system of example 2, wherein the plurality of extracted features comprise a plurality of correlation coefficients and a plurality of R-R intervals.

Example 4: The medical system of example 3, wherein the plurality of correlation coefficients comprises three correlation coefficients and the plurality of R-R intervals includes four R-R intervals.

Example 5: The medical system of example 4, wherein the plurality of extracted features further comprises one or more of a maximum QRS amplitude, a minimum QRS amplitude, an amplitude difference between the maximum QRS amplitude and the minimum QRS amplitude, a number of samples between the maximum QRS amplitude and the minimum QRS amplitude, a maximum slope of the QRS wave, a minimum slope of the QRS wave, a difference of slope value between the maximum slope of the QRS wave and the minimum slope of the QRS wave, or a number of samples between the maximum slope of the QRS wave and the minimum slope of the QRS wave.

Example 6: The medical system of example 5, wherein the processing circuitry is further configured to: adjust a size of the image of the sensed cardiac electrogram corresponding to a respective cardiac electrogram weight; adjust a size the feature images corresponding to a respective image weight; and apply the machine learning model to the size-adjusted image of the sensed cardiac electrogram and the size-adjusted feature images to determine whether one or more particular heartbeats in the sensed cardiac electrogram is a PVC beat.

Example 7: The medical system of example 6, wherein the processing circuitry is further configured to: normalize axes of the image of the sensed cardiac electrogram; normalize the feature images; and apply the machine learning model to the normalized image of the sensed cardiac electrogram and the normalized feature images to determine whether one or more particular heartbeats in the sensed cardiac electrogram is a PVC beat.

Example 8: The medical system of any of examples 6 and 7, wherein the image of the sensed cardiac electrogram is weighted more than the feature images.

Example 9: The medical system of example 8, wherein the feature image corresponding to the plurality of extracted correlation coefficients is weighted more than one or more of the feature images for the maximum QRS amplitude, the minimum QRS amplitude, the amplitude difference between the maximum QRS amplitude and the minimum QRS amplitude, the number of samples between the maximum QRS amplitude and the minimum QRS amplitude, the maximum slope of the QRS wave, the minimum slope of the QRS wave, the difference of slope value between the maximum slope of the QRS wave and the minimum slope of the QRS wave, and the number of samples between the maximum slope of the QRS wave and the minimum slope of the QRS, and the feature image corresponding to the extracted plurality of R-R intervals is weighted more than each of the feature images for the maximum QRS amplitude, the minimum QRS amplitude, the amplitude difference between the maximum QRS amplitude and the minimum QRS amplitude, the number of samples between the maximum QRS amplitude and the minimum QRS amplitude, the maximum slope of the QRS wave, the minimum slope of the QRS wave, the difference of slope value between the maximum slope of the QRS wave and the minimum slope of the QRS wave, and the number of samples between the maximum slope of the QRS wave and the minimum slope of the QRS.

Example 10: The medical system of any of examples 1 through 9, wherein the processing circuitry is further configured to apply the machine learning model to the image of the sensed cardiac electrogram and the one or more feature images to determine a location of origin of a PVC beat.

Example 11: The medical system of any of examples 1 through 10, wherein the medical system is an insertable cardiac monitor, the insertable cardiac monitor includes a power source operatively coupled to the processing circuitry; a memory operatively coupled to the processing circuitry and configured to store the machine learning model; a distal electrode operatively coupled to the processing circuitry; a proximal electrode operatively coupled to the processing circuitry; and a hermetically-sealed housing configured for subcutaneous implantation within the patient, wherein at least the power source, memory, and processing circuitry are within the hermetically-sealed case, and wherein the housing has a length, a width, and a depth, wherein the length is greater than the width and the width is greater than the depth, wherein the length is within a range from 5 millimeters (mm) to 60 mm, wherein the width is within a range from 5 mm to 15 mm, and wherein the depth is within a range from 5 mm to 15 mm.

Example 12: The medical system of any of examples 1 through 11, wherein prior to the machine learning model being applied to the respective feature image, the machine learning model was trained by: selection of a training set comprising a set of training instances, each training instance comprising an association between one or more respective cardiac electrogram features of a respective feature image and a respective PVC beat; and for each training instance in the training set, modification, based on the respective cardiac electrogram features and the respective PVC beat of the training instance, of the machine learning model to change a score generated by the machine learning model in response to subsequent PVC beats applied to the machine learning model.

Example 13: A computing device includes a memory; and processing circuitry coupled to the memory, the processing circuitry being configured to: receive a sensed cardiac electrogram of a patient; perform feature extraction of the sensed cardiac electrogram to extract a plurality of features from the sensed cardiac electrogram; convert one or more of the plurality of extracted features to a respective feature image; apply a machine learning model, trained using cardiac electrogram data for a plurality of patients, to an image of the sensed cardiac electrogram and at least the respective feature image to determine whether one or more particular heartbeats in the sensed cardiac electrogram indicate a premature ventricular contraction (PVC) beat; and in response to the determination that the one or more particular heartbeats indicates a PVC beat, output a classification that the one or more particular heartbeats is a PVC beat.

Example 14: The computing device of example 13, wherein the sensed cardiac electrogram comprises signals of consecutive heartbeats, the signals of the consecutive heartbeats including a signal of the one or more particular heartbeats, a signal of a heartbeat immediately preceding the one or more particular heartbeats and a signal of a heartbeat immediately after the one or more particular heartbeats.

Example 15: The computing device of example 14, wherein the plurality of extracted features comprise a plurality of correlation coefficients and a plurality of R-R intervals.

Example 16: The computing device of example 15, wherein the plurality of correlation coefficients comprises three correlation coefficients and the plurality of R-R intervals includes four R-R intervals.

Example 17: The computing device of example 16, wherein the plurality of extracted features further comprises one or more of a maximum QRS amplitude, a minimum QRS amplitude, an amplitude difference between the maximum QRS amplitude and the minimum QRS amplitude, a number of samples between the maximum QRS amplitude and the minimum QRS amplitude, a maximum slope of the QRS wave, a minimum slope of the QRS wave, a difference of slope value between the maximum slope of the QRS wave and the minimum slope of the QRS wave, or a number of samples between the maximum slope of the QRS wave and the minimum slope of the QRS wave.

Example 18: The computing device of example 17, wherein the processing circuitry is further configured to: adjust a size of the image of the sensed cardiac electrogram corresponding to a respective cardiac electrogram weight; adjust a size the feature images corresponding to a respective image weight; apply the machine learning model to the size-adjusted image of the sensed cardiac electrogram and the size-adjusted feature images to determine whether one or more particular heartbeats in the sensed cardiac electrogram is a PVC beat.

Example 19: The computing device of example 18, wherein the processing circuitry is further configured to: normalize axes of the image of the sensed cardiac electrogram; normalize the feature images; and apply the machine learning model to the normalized image of the sensed cardiac electrogram and the normalized feature images to determine whether one or more particular heartbeats in the sensed cardiac electrogram is a PVC beat.

Example 20: The computing device of any of examples 18 and 19, wherein the image of the sensed cardiac electrogram is weighted more than the feature images.

Example 21: The computing device of example 20, wherein the feature image corresponding to the plurality of extracted correlation coefficients is weighted more than one or more of the feature images for the maximum QRS amplitude, the minimum QRS amplitude, the amplitude difference between the maximum QRS amplitude and the minimum QRS amplitude, the number of samples between the maximum QRS amplitude and the minimum QRS amplitude, the maximum slope of the QRS wave, the minimum slope of the QRS wave, the difference of slope value between the maximum slope of the QRS wave and the minimum slope of the QRS wave, and the number of samples between the maximum slope of the QRS wave and the minimum slope of the QRS, and the number of samples between the maximum slope of the QRS wave and the minimum slope of the QRS.

Example 22: The computing device of any of examples 13 through 21, wherein the processing circuitry is further configured to apply the machine learning model to the image of the sensed cardiac electrogram and the one or more feature images to determine a location of origin of a PVC beat.

Example 23: The computing device of any of examples 13 through 22, wherein prior to the machine learning model being applied to the respective feature image, the machine learning model was trained by: selection of a training set comprising a set of training instances, each training instance comprising an association between one or more respective cardiac electrogram features of a respective feature image and a respective PVC beat; and for each training instance in the training set, modification, based on the respective cardiac electrogram features and the respective PVC beat of the training instance, of the machine learning model to change a score generated by the machine learning model in response to subsequent PVC beats applied to the machine learning model.

Example 24: A method includes receiving a sensed cardiac electrogram of a patient; performing feature extraction of the sensed cardiac electrogram to extract a plurality of features from the sensed cardiac electrogram; converting one or more of the plurality of extracted features to a respective feature image; applying a machine learning model, trained using cardiac electrogram data for a plurality of patients, to an image of the sensed cardiac electrogram and at least the respective feature image to determine whether one or more particular heartbeats in the sensed cardiac electrogram indicate a premature ventricular contraction (PVC) beat; and in response to the determination that the one or more particular heartbeats indicates a PVC beat, outputting a classification that the one or more particular heartbeats is a PVC beat.

Example 25: The method of example 24, wherein the sensed cardiac electrogram comprises signals of consecutive heartbeats, the signals of the consecutive heartbeats including a signal of the one or more particular heartbeats, a signal of a heartbeat immediately preceding the one or more particular heartbeats and a signal of a heartbeat immediately after the one or more particular heartbeats.

Example 26: The method of example 25, wherein the plurality of extracted features comprise a plurality of correlation coefficients and a plurality of R-R intervals.

Example 27: The method of example 26, wherein the plurality of correlation coefficients comprises three correlation coefficients and the plurality of R-R intervals includes four R-R intervals.

Example 28: The method of example 27, wherein the plurality of extracted features further comprises one or more of a maximum QRS amplitude, a minimum QRS amplitude, an amplitude difference between the maximum QRS amplitude and the minimum QRS amplitude, a number of samples between the maximum QRS amplitude and the minimum QRS amplitude, a maximum slope of the QRS wave, a minimum slope of the QRS wave, a difference of slope value between the maximum slope of the QRS wave and the minimum slope of the QRS wave, or a number of samples between the maximum slope of the QRS wave and the minimum slope of the QRS wave.

Example 29: The method of example 28, wherein the method further comprises: adjusting a size of the image of the sensed cardiac electrogram corresponding to a respective cardiac electrogram weight; adjusting a size the feature images corresponding to a respective image weight; and applying the machine learning model to the size-adjusted image of the sensed cardiac electrogram and the size-adjusted feature images to determine whether one or more particular heartbeats in the sensed cardiac electrogram is a PVC beat.

Example 30: The method of example 29, wherein the method further comprises: normalizing axes of the image of the sensed cardiac electrogram; normalizing the feature images; and applying the machine learning model to the normalized image of the sensed cardiac electrogram and the normalized feature images to determine whether one or more particular heartbeats in the sensed cardiac electrogram is a PVC beat.

Example 31: The method of any of examples 29 and 30, wherein the image of the sensed cardiac electrogram is weighted more than the feature images.

Example 32: The method of example 31, wherein the feature image corresponding to the plurality of extracted correlation coefficients is weighted more than one or more of the feature images for the maximum QRS amplitude, the minimum QRS amplitude, the amplitude difference between the maximum QRS amplitude and the minimum QRS amplitude, the number of samples between the maximum QRS amplitude and the minimum QRS amplitude, the maximum slope of the QRS wave, the minimum slope of the QRS wave, the difference of slope value between the maximum slope of the QRS wave and the minimum slope of the QRS wave, and the number of samples between the maximum slope of the QRS wave and the minimum slope of the QRS, and the number of samples between the maximum slope of the QRS wave and the minimum slope of the QRS.

Example 33: The method of any of examples 24 through 32, wherein the method further comprises: applying the machine learning model to the image of the sensed cardiac electrogram and the one or more feature images to determine a location of origin of a PVC beat.

Various examples have been described. These and other examples are within the scope of the following claims.

What is claimed is:

1. A medical system comprising:
a plurality of electrodes configured to sense a cardiac electrogram of a patient; and
processing circuitry configured to:
perform feature extraction of the sensed cardiac electrogram to extract a plurality of features from the sensed cardiac electrogram;
convert one or more of the plurality of extracted features to a respective feature image;
apply a machine learning model, trained using cardiac electrogram data for a plurality of patients, to an image of the sensed cardiac electrogram and at least the respective feature image to determine whether one or more particular heartbeats in the sensed cardiac electrogram indicate a premature ventricular contraction (PVC) beat; and
in response to the determination that the one or more particular heartbeats indicates a PVC beat, output a classification that the one or more particular heartbeats is a PVC beat.

2. The medical system of claim 1, wherein the sensed cardiac electrogram comprises signals of consecutive heartbeats, the signals of the consecutive heartbeats including a signal of the one or more particular heartbeats, a signal of a heartbeat immediately preceding the one or more particular heartbeats and a signal of a heartbeat immediately after the one or more particular heartbeats.

3. The medical system of claim 2, wherein the plurality of extracted features comprise a plurality of correlation coefficients and a plurality of R-R intervals.

4. The medical system of claim 3, wherein the plurality of correlation coefficients comprises three correlation coefficients and the plurality of R-R intervals includes four R-R intervals.

5. The medical system of claim 4, wherein the plurality of extracted features further comprises one or more of a maximum QRS amplitude, a minimum QRS amplitude, an amplitude difference between the maximum QRS amplitude and the minimum QRS amplitude, a number of samples between the maximum QRS amplitude and the minimum QRS amplitude, a maximum slope of the QRS wave, a minimum slope of the QRS wave, a difference of slope value between the maximum slope of the QRS wave and the minimum slope of the QRS wave, or a number of samples between the maximum slope of the QRS wave and the minimum slope of the QRS wave.

6. The medical system of claim 5, wherein the processing circuitry is further configured to:
adjust a size of the image of the sensed cardiac electrogram corresponding to a respective cardiac electrogram weight;
adjust a size the feature images corresponding to a respective image weight; and
apply the machine learning model to the size-adjusted image of the sensed cardiac electrogram and the size-adjusted feature images to determine whether one or more particular heartbeats in the sensed cardiac electrogram is a PVC beat.

7. The medical system of claim 6, wherein the processing circuitry is further configured to:
normalize axes of the image of the sensed cardiac electrogram;
normalize the feature images; and
apply the machine learning model to the normalized image of the sensed cardiac electrogram and the normalized feature images to determine whether one or more particular heartbeats in the sensed cardiac electrogram is a PVC beat.

8. The medical system of claim 6, wherein the image of the sensed cardiac electrogram is weighted more than the feature images.

9. The medical system of claim 8, wherein the feature image corresponding to the plurality of extracted correlation coefficients is weighted more than one or more of the feature images for the maximum QRS amplitude, the minimum QRS amplitude, the amplitude difference between the maximum QRS amplitude and the minimum QRS amplitude, the number of samples between the maximum QRS amplitude and the minimum QRS amplitude, the maximum slope of the QRS wave, the minimum slope of the QRS wave, the difference of slope value between the maximum slope of the QRS wave and the minimum slope of the QRS wave, and the number of samples between the maximum slope of the QRS wave and the minimum slope of the QRS, and
the feature image corresponding to the extracted plurality of R-R intervals is weighted more than each of the feature images for the maximum QRS amplitude, the minimum QRS amplitude, the amplitude difference between the maximum QRS amplitude and the minimum QRS amplitude, the number of samples between the maximum QRS amplitude and the minimum QRS amplitude, the maximum slope of the QRS wave, the minimum slope of the QRS wave, the difference of slope value between the maximum slope of the QRS wave and the minimum slope of the QRS wave, and the number of samples between the maximum slope of the QRS wave and the minimum slope of the QRS.

10. The medical system of claim 1, wherein the processing circuitry is further configured to apply the machine learning model to the image of the sensed cardiac electrogram and the one or more feature images to determine a location of origin of a PVC beat.

11. The medical system of claim 1, wherein the medical system is an insertable cardiac monitor, the insertable cardiac monitor comprising:
a power source operatively coupled to the processing circuitry;
a memory operatively coupled to the processing circuitry and configured to store the machine learning model;
a distal electrode operatively coupled to the processing circuitry;
a proximal electrode operatively coupled to the processing circuitry; and
a hermetically-sealed housing configured for subcutaneous implantation within the patient, wherein at least the power source, memory, and processing circuitry are within the hermetically-sealed case, and
wherein the housing has a length, a width, and a depth,
wherein the length is greater than the width and the width is greater than the depth,
wherein the length is within a range from 5 millimeters (mm) to 60 mm,
wherein the width is within a range from 5 mm to 15 mm, and
wherein the depth is within a range from 5 mm to 15 mm.

12. The medical system of claim 1, wherein prior to the machine learning model being applied to the respective feature image, the machine learning model was trained by: selection of a training set comprising a set of training instances, each training instance comprising an association between one or more respective cardiac electrogram features of a respective feature image and a respective PVC beat; and for each training instance in the training set, modification, based on the respective cardiac electrogram features and the respective PVC beat of the training instance, of the machine learning model to change a score generated by the machine learning model in response to subsequent PVC beats applied to the machine learning model.

13. A computing device comprising:
a memory; and
processing circuitry coupled to the memory, the processing circuitry being configured to:
receive a sensed cardiac electrogram of a patient;
perform feature extraction of the sensed cardiac electrogram to extract a plurality of features from the sensed cardiac electrogram;
convert one or more of the plurality of extracted features to a respective feature image;
apply a machine learning model, trained using cardiac electrogram data for a plurality of patients, to an image of the sensed cardiac electrogram and at least the respective feature image to determine whether one or more particular heartbeats in the sensed cardiac electrogram indicate a premature ventricular contraction (PVC) beat; and
in response to the determination that the one or more particular heartbeats indicates a PVC beat, output a classification that the one or more particular heartbeats is a PVC beat.

14. The computing device of claim 13, wherein the sensed cardiac electrogram comprises signals of consecutive heartbeats, the signals of the consecutive heartbeats including a signal of the one or more particular heartbeats, a signal of a heartbeat immediately preceding the one or more particular heartbeats and a signal of a heartbeat immediately after the one or more particular heartbeats.

15. The computing device of claim 14, wherein the plurality of extracted features comprise a plurality of correlation coefficients and a plurality of R-R intervals.

16. The computing device of claim 15, wherein the plurality of correlation coefficients comprises three correlation coefficients and the plurality of R-R intervals includes four R-R intervals.

17. The computing device of claim 16, wherein the plurality of extracted features further comprises one or more of a maximum QRS amplitude, a minimum QRS amplitude, an amplitude difference between the maximum QRS amplitude and the minimum QRS amplitude, a number of samples between the maximum QRS amplitude and the minimum QRS amplitude, a maximum slope of the QRS wave, a minimum slope of the QRS wave, a difference of slope value between the maximum slope of the QRS wave and the minimum slope of the QRS wave, or a number of samples between the maximum slope of the QRS wave and the minimum slope of the QRS wave.

18. The computing device of claim 17, wherein the processing circuitry is further configured to:
adjust a size of the image of the sensed cardiac electrogram corresponding to a respective cardiac electrogram weight;
adjust a size the feature images corresponding to a respective image weight;
apply the machine learning model to the size-adjusted image of the sensed cardiac electrogram and the size-adjusted feature images to determine whether one or more particular heartbeats in the sensed cardiac electrogram is a PVC beat.

19. The computing device of claim 18, wherein the processing circuitry is further configured to:
normalize axes of the image of the sensed cardiac electrogram;
normalize the feature images; and
apply the machine learning model to the normalized image of the sensed cardiac electrogram and the normalized feature images to determine whether one or more particular heartbeats in the sensed cardiac electrogram is a PVC beat.

20. The computing device of claim 18, wherein the image of the sensed cardiac electrogram is weighted more than the feature images.

21. The computing device of claim 20, wherein the feature image corresponding to the plurality of extracted correlation coefficients is weighted more than one or more of the feature images for the maximum QRS amplitude, the minimum QRS amplitude, the amplitude difference between the maximum QRS amplitude and the minimum QRS amplitude, the number of samples between the maximum QRS amplitude and the minimum QRS amplitude, the maximum slope of the QRS wave, the minimum slope of the QRS wave, the difference of slope value between the maximum slope of the QRS wave and the minimum slope of the QRS wave, and the number of samples between the maximum slope of the QRS wave and the minimum slope of the QRS, and the number of samples between the maximum slope of the QRS wave and the minimum slope of the QRS.

22. The computing device of claim 13, wherein the processing circuitry is further configured to apply the machine learning model to the image of the sensed cardiac electrogram and the one or more feature images to determine a location of origin of a PVC beat.

23. The computing device of claim 13, wherein prior to the machine learning model being applied to the respective feature image, the machine learning model was trained by: selection of a training set comprising a set of training instances, each training instance comprising an association between one or more respective cardiac electrogram features of a respective feature image and a respective PVC beat; and for each training instance in the training set, modification, based on the respective cardiac electrogram features and the respective PVC beat of the training instance, of the machine learning model to change a score generated by the machine learning model in response to subsequent PVC beats applied to the machine learning model.

24. A method comprising:
receiving a sensed cardiac electrogram of a patient;
performing feature extraction of the sensed cardiac electrogram to extract a plurality of features from the sensed cardiac electrogram;
converting one or more of the plurality of extracted features to a respective feature image;
applying a machine learning model, trained using cardiac electrogram data for a plurality of patients, to an image of the sensed cardiac electrogram and at least the respective feature image to determine whether one or more particular heartbeats in the sensed cardiac electrogram indicate a premature ventricular contraction (PVC) beat; and
in response to the determination that the one or more particular heartbeats indicates a PVC beat, outputting a classification that the one or more particular heartbeats is a PVC beat.

25. The method of claim 24, wherein the sensed cardiac electrogram comprises signals of consecutive heartbeats, the signals of the consecutive heartbeats including a signal of the one or more particular heartbeats, a signal of a heartbeat immediately preceding the one or more particular heartbeats and a signal of a heartbeat immediately after the one or more particular heartbeats.

26. The method of claim 25, wherein the plurality of extracted features comprise a plurality of correlation coefficients and a plurality of R-R intervals.

27. The method of claim 26, wherein the plurality of correlation coefficients comprises three correlation coefficients and the plurality of R-R intervals includes four R-R intervals.

28. The method of claim 27, wherein the plurality of extracted features further comprises one or more of a maximum QRS amplitude, a minimum QRS amplitude, an amplitude difference between the maximum QRS amplitude and the minimum QRS amplitude, a number of samples between the maximum QRS amplitude and the minimum QRS amplitude, a maximum slope of the QRS wave, a minimum slope of the QRS wave, a difference of slope value between the maximum slope of the QRS wave and the minimum slope of the QRS wave, or a number of samples between the maximum slope of the QRS wave and the minimum slope of the QRS wave.

29. The method of claim 28, wherein the method further comprises:
adjusting a size of the image of the sensed cardiac electrogram corresponding to a respective cardiac electrogram weight;
adjusting a size the feature images corresponding to a respective image weight; and
applying the machine learning model to the size-adjusted image of the sensed cardiac electrogram and the size-adjusted feature images to determine whether one or more particular heartbeats in the sensed cardiac electrogram is a PVC beat.

30. The method of claim 29, wherein the method further comprises:
normalizing axes of the image of the sensed cardiac electrogram;
normalizing the feature images; and
applying the machine learning model to the normalized image of the sensed cardiac electrogram and the normalized feature images to determine whether one or more particular heartbeats in the sensed cardiac electrogram is a PVC beat.

31. The method of claim 29, wherein the image of the sensed cardiac electrogram is weighted more than the feature images.

32. The method of claim 31, wherein the feature image corresponding to the plurality of extracted correlation coefficients is weighted more than one or more of the feature images for the maximum QRS amplitude, the minimum QRS amplitude, the amplitude difference between the maximum QRS amplitude and the minimum QRS amplitude, the number of samples between the maximum QRS amplitude and the minimum QRS amplitude, the maximum slope of the QRS wave, the minimum slope of the QRS wave, the difference of slope value between the maximum slope of the QRS wave and the minimum slope of the QRS wave, and the number of samples between the maximum slope of the QRS wave and the minimum slope of the QRS, and the number of samples between the maximum slope of the QRS wave and the minimum slope of the QRS.

33. The method of claim 24, wherein the method further comprises:
applying the machine learning model to the image of the sensed cardiac electrogram and the one or more feature images to determine a location of origin of a PVC beat.
